(19) United States
(12) Patent Application Publication
(10) Pub. No.: US 2025/0241685 A1
(43) Pub. Date: Jul. 31, 2025

(54) MODULAR SCREW FOR A VERTEBRAL FIXATION SYSTEM

(71) Applicant: Highridge Medical, LLC, Westminster, CO (US)

(72) Inventors: Douglas Brooke, Golden, CO (US); Guillaume Quetier, Lakewood, CO (US)

(21) Appl. No.: 19/036,932

(22) Filed: Jan. 24, 2025

Related U.S. Application Data
(60) Provisional application No. 63/625,170, filed on Jan. 25, 2024.

(51) Int. Cl. A61B 17/70; A61B 17/00

(52) U.S. Cl. CPC A61B 17/7032; A61B 17/7037; A61B 2017/00477

(57) ABSTRACT

A modular screw for a vertebral fixation system includes a fastener with a head and shank, and a housing assembly including a housing and a piston. The head is insertable via bottom-loading into a distal end of the housing. The housing assembly may include a ring and one or more biasing members, where the piston is downward biased by the one or more biasing members and holds the ring in a first position. The head engages the piston and causes the piston to release the ring, which transitions to a second position and engages the head and prevents removal of the head from the housing. The one or more biasing members include a spring that is optionally integrally formed with the piston. In addition, the piston is optionally a multi-piece component. Further, the head is optionally a multi-piece component, with a portion installed within the housing via a shear pin.

### CROSS-REFERENCE TO RELATED APPLICATIONS

This U.S. Non-Provisional Patent application claims the benefit of priority under 35 U.S.C. § 119 (e) of U.S. Provisional Patent Application Ser. No. 63/625,170, filed Jan. 25, 2024, the entire disclosure of which is hereby incorporated by reference.

### FIELD OF THE DISCLOSURE

The disclosure relates to an osseous screw and, in particular, is directed to a modular screw for a vertebral fixation system, and method of installation of the same.

### BACKGROUND

The background description includes information that may be useful in understanding the present inventive subject matter. It is not an admission that any of the information provided herein is prior art or relevant to the presently claimed disclosure, or that any publication specifically or implicitly referenced is prior art.

Vertebral fixation systems include screws that are inserted into vertebrae. The screws are able to receive stabilization members that secure the vertebrae together. In some configurations of vertebral fixation systems, the screws include multiple components such as a fastener and a housing. In these configurations, a distal tip of a shank of the fastener is insertable into a proximal or top end of the housing. The shank passes through the housing until a head of the fastener rests within a distal end of a channel (and/or cavity) of the housing.

### SUMMARY

The top-loaded configurations for vertebral fixation screws as described above have shortcomings, including the need to align the fastener within the vertebrae after the entire screw is assembled. This can increase the complexity of inserting the screw into the vertebrae by a tool, as the tool may need to engage the housing (i.e., an exterior surface or surfaces) to cause the screw to turn and/or needs to be insertable into the housing to engage a tool interface in the head of the fastener. This can be especially problematic in minimally invasive vertebral fixation systems, where an increase in tool size and/or complexity of motion during installation generally results in an increase in the size of the surgical site.

As such, there exists a need for a modular screw that includes a fastener able to be bottom-loaded into a housing. Embodiments of the present disclosure are directed to a modular screw with a housing assembly and a fastener. The fastener should include a head, and a shank threadable into a vertebra. Embodiments of the present disclosure are also directed to the head being able to receive a housing of the modular screw after the fastener is inserted into the vertebrae, which couples to the head following the head passing through an aperture in a distal end of the housing (i.e., such that the head is bottom loaded into the housing). After the head is inserted into the distal end of the housing, components within the housing engage with the head and prevent the removal of the head from the distal end of the housing. In this regard, the complexity of the vertebral fixation system is reduced, as the need for a tool that is able to interface with the housing and/or pass through the housing to turn the fastener into the vertebrae is reduced or eliminated. This additionally allows for a smaller tool, and further allows for a reduced size of surgical site and impact on a patient, especially in minimally invasive systems.

Embodiments of the present disclosure are further directed to biasing members or biasing elements utilized to actuate components within the housing, such as a piston and a ring. In particular, engagement with a biased piston to cause a transition toward a proximal end of the housing releases a ring, where the ring transitions to a position within the housing to engage with the head of the fastener. Thus, removal of the fastener from the distal end of the housing is prevented by the engagement of the ring to the head.

Embodiments of the present disclosure are further directed to the spring being integrally formed with the piston, and/or are directed to the piston being a single-piece component or a multi-piece component or assembly.

Embodiments of the present disclosure are further directed to the head of the fastener having a first portion that is coupled to (or integrated with) the shank of the fastener, and a second portion that is pre-installed within the housing (and thus the housing assembly). The second portion of the head of the fastener is held within the housing by a pin. Optionally, the pin is shearable with the application of a pre-determined amount of force. Prior to the application of the pre-determined amount of force, the second portion of the head of the fastener (and the first portion/shank of the fastener, when coupled to the second portion) is set at a particular orientation relative to the housing, preventing polyaxial movement of the housing about the head of the fastener. After the pre-determined force is applied, the pin is sheared or otherwise broken and the housing is allowed to have polyaxial movement about the head of the fastener.

Embodiments are further directed to an interior surface of the housing and/or an exterior surface of the piston being tapered. The tapering of the interior surface of the housing and/or an exterior surface of the piston reduces or prevents polyaxial movement of the piston relative to the housing.

Embodiments of the present disclosure are further directed to a tool pin that engages the piston. The tool pin passes through an aperture in the housing and protrudes into a tool engagement feature in an arm of the housing. Engagement by the tool with the tool engagement feature will also result in engagement of the tool pin, preventing polyaxial movement of the piston relative to the housing.

A first aspect of the present disclosure is to provide a modular screw for a vertebral fixation system. The modular screw includes a housing assembly and a fastener. The housing assembly includes a housing with a head cavity, a piston cavity, and a channel to receive a stabilization member; a piston installed in the piston cavity and at least partially extend into the head cavity; a ring installed within the head cavity; and one or more biasing members that bias the piston toward the ring and into the head cavity. The fastener includes a shank and a head, wherein the head is insertable into the head cavity via a bottom-loading of the head into the head cavity through a head aperture at a distal end of the housing.

The modular screw of the first aspect may include, optionally, wherein the one or more biasing members causes the piston to be biased toward the distal end of the housing absent an application of force to the piston by the head of the fastener, wherein the ring is held in a first position by the piston prior to an application of force by the head on the piston, wherein the ring is released by the piston and engages the head following an application of force by the head on the piston, and wherein the ring engaging the head prevents removal of the head from the distal end of the housing.

The modular screw of the first aspect may include one or more of the previous embodiments and, optionally, wherein the one or more biasing members comprises a pin extending into the housing and a spring that engages the piston.

The modular screw of the first aspect may include one or more of the previous embodiments and, optionally, wherein the spring of the one or more biasing members are integrally formed with the piston.

The modular screw of the first aspect may include one or more of the previous embodiments and, optionally, wherein the piston comprises: a first portion to engage with the one or more biasing members; and a second portion to engage with at least one of the ring and the head.

The modular screw of the first aspect may include one or more of the previous embodiments and, optionally, wherein the ring is a c-ring.

The modular screw of the first aspect may include one or more of the previous embodiments and, optionally, wherein the modular screw is configured for polyaxial movement of the head of the fastener relative to the housing.

The modular screw of the first aspect may include one or more of the previous embodiments and, optionally, wherein the modular screw is configured for unidirectional movement of the head of the fastener relative to the housing.

The modular screw of the first aspect may include one or more of the previous embodiments and, optionally, wherein the ring includes one or more trunnions when the modular screw is configured for unidirectional movement.

The modular screw of the first aspect may include one or more of the previous embodiments and, optionally, a locking assembly including one or more members within respective member channels in the housing; and one or more pins that correspond to a particular member of the one or more members. A force applied to a particular member of the one or more members is transferred through the corresponding pin of the one or more pins to the piston. The force being applied to the piston prevents rotation in one or more directions of the head of the fastener relative to the housing.

The modular screw of the first aspect may include one or more of the previous embodiments and, optionally, may be usable as a component of the vertebral fixation system with at least one of a set screw and the stabilization member.

The modular screw of the first aspect may include one or more of the previous embodiments and, optionally, being installable as part of the vertebral fixation system via a method or process directed at least to: insertion of the head via the bottom-loading into the distal end of the housing; and engagement of the piston by the head to cause the ring to engage the head and prevent removal of the head from the distal end of the housing.

A second aspect of the present disclosure is to provide to a modular screw for a vertebral fixation system. The modular screw includes a housing assembly and a fastener. The housing assembly includes a housing with a base and at least one arm, wherein the base and the at least one arm define a head cavity, a piston cavity, and a channel able to receive a stabilization member; a piston installed within the piston cavity and at least partially extending into the head cavity; a ring installed within the head cavity; and one or more biasing members that bias the piston toward the ring and into the head cavity. The fastener includes a shank and a head, wherein the head is insertable into the head cavity via a bottom-loading of the head into the head cavity through a head aperture at a distal end of the housing.

The modular screw of the second aspect may include, optionally, wherein the one or more biasing members causes the piston to be biased toward the distal end of the housing absent an application of force to the piston by the head of the fastener, wherein the ring is held in a first position by the piston prior to an application of force by the head on the piston, wherein the ring is released by the piston following an application of force by the head on the piston and engages the head, and wherein the ring engaging the head prevents removal of the head from the distal end of the housing.

The modular screw of the second aspect may include one or more of the previous embodiments and, optionally, wherein the one or more biasing members comprises a pin extending into the housing and a spring that engages the piston.

The modular screw of the second aspect may include one or more of the previous embodiments and, optionally, wherein the one or more biasing members comprises a spring inserted within the housing and engages the piston, wherein the spring is inserted at an oblique angle relative to a longitudinal axis through the housing.

The modular screw of the second aspect may include one or more of the previous embodiments and, optionally, wherein the piston comprises: a first portion to engage with the one or more biasing members; and a second portion to engage with at least one of the ring and the head.

The modular screw of the second aspect may include one or more of the previous embodiments and, optionally, wherein the ring is a c-ring.

The modular screw of the second aspect may include one or more of the previous embodiments and, optionally, wherein the modular screw is configured for polyaxial movement of the housing relative to the head of the fastener.

The modular screw of the second aspect may include one or more of the previous embodiments and, optionally, wherein the modular screw is configured for unidirectional movement of the housing relative to the head of the fastener.

The modular screw of the second aspect may include one or more of the previous embodiments and, optionally, wherein the ring includes one or more trunnions when the modular screw is configured for unidirectional movement.

The modular screw of the second aspect may include one or more of the previous embodiments and, optionally, wherein the modular screw includes a locking assembly. The locking assembly includes one or more members within respective member channels in the housing; and one or more pins that correspond to a particular member of the one or more members, wherein a force applied to a particular member of the one or more members is transferred through the corresponding pin of the one or more pins to the piston, and wherein the force being applied to the piston prevents rotation in one or more directions of the head of the fastener relative to the housing.

The modular screw of the second aspect may include one or more of the previous embodiments and, optionally, being usable as a component of the vertebral fixation system with at least one of a set screw and a stabilization member that are each insertable into the housing.

A third aspect of the present disclosure is to provide a modular screw for a vertebral fixation system. The modular screw includes a fastener and a housing assembly. The fastener includes a shank and a head, the head including a first portion. The housing assembly includes a housing with a base and at least one arm, wherein the base and the at least one arm define a head cavity, a piston cavity, and a channel able to receive a stabilization member; a piston installed within the piston cavity and at least partially extending into the head cavity; a second portion of the head of the fastener within the head cavity; and a pin able to secure the second portion of the head of the fastener at a particular orientation within the head cavity relative to the housing. The first portion of the head is insertable into the second portion of the head within the housing assembly via a bottom-loading of the first portion of the head into the head cavity through a head aperture at a distal end of the housing, wherein polyaxial movement of the housing about the head of the fastener is reduced by the pin when the pin engages the housing and the second portion of the head of the fastener, and wherein the pin is able to be sheared at a shear point with a pre-determined amount of force to allow for polyaxial movement of the housing about the head of the fastener.

The modular screw of the third aspect may include, optionally, wherein the base and the at least one arm of the housing further define a piston cavity, and wherein the housing assembly further comprises a tool pin in engagement with a pin aperture in the piston, wherein the tool pin is located within a tool engagement feature in the at least one arm of the housing.

The modular screw of the third aspect may include one or more of the previous embodiments and, optionally, wherein the tool pin is affixed to the pin aperture of the piston.

The modular screw the third aspect may include one or more of the previous embodiments and, optionally, wherein at least one of an interior surface of the housing and an exterior surface of the piston is tapered to reduce polyaxial movement of the piston relative to the housing.

A fourth aspect of the present disclosure is to provide a method. The method optionally includes, but is not limited to, providing a housing assembly and a fastener of a modular screw, the housing assembly comprising a piston within a housing including a base and at least one arm that define a head cavity, a piston cavity, and a channel able to receive a stabilization member, the fastener comprising a shank and a head. The method optionally includes, but is not limited to, inserting at least a portion of the head of the fastener into the head cavity of the housing via bottom-loading into a head aperture at a distal end of the housing. Engagement of interlocking features within the head cavity of the housing post-insertion of the at least a portion of the head of the fastener into the head cavity of the housing prevents removal of the head from the distal end of the housing.

The method of the fourth aspect optionally includes, but is not limited to, actuating the piston with the head toward a proximal end of the housing relative to a position pre-set by one or more biasing members that engage with and bias the piston toward the distal end of the housing, wherein the actuating of the piston with the head releases a ring installed within the head cavity and into the head cavity, and wherein the released ring engages with the head to prevent removal of the head from the distal end of the housing.

The method of the fourth aspect optionally includes one or more of the previous embodiments, and further may include, but is not limited to, providing a pre-assembly of the modular screw, the pre-assembly including the housing, the piston, a second portion of the fastener that is able to receive and engage with a first portion of the fastener, and a pin able to secure the second portion of the head of the fastener at a particular orientation within the head cavity relative to the housing. The method optionally includes, but is not limited to, coupling the first portion of the head of the fastener into the second portion of the head of the fastener within the head cavity of the housing via threading that prevents removal of the head from the distal end of the housing.

The method of the fourth aspect optionally includes one or more of the previous embodiments, and further may include, but is not limited to, applying a pre-determined amount of force to the modular screw to shear the pin at a shear point and allow for polyaxial movement of the housing about the head of the fastener.

The method of the fourth aspect optionally includes one or more of the previous embodiments, and further may include, but is not limited to, engaging a tool pin coupled to the piston and accessible via a tool engagement feature in the at least one arm of the housing, wherein engagement of the tool pin prevents polyaxial movement of the piston relative to the housing.

The phrases “at least one,” “one or more,” and “and/or,” as used herein, are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together.

Unless otherwise indicated, all numbers expressing quantities, dimensions, conditions, and so forth used in the specification and claims are to be understood as being modified in all instances by the term “about.” As used herein, unless otherwise specified, the terms “about,” “approximately,” etc., when used in relation to numerical limitations or ranges, mean that the recited limitation or range may vary by up to 10%. By way of non-limiting example, “about 750” can mean as little as 675 or as much as 825, or any value therebetween. When used in relation to ratios or relationships between two or more numerical limitations or ranges, the terms “about,” “approximately,” etc. mean that each of the limitations or ranges may vary by up to 10%; by way of non-limiting example, a statement that two quantities are “approximately equal” can mean that a ratio between the two quantities is as little as 0.9:1.1 or as much as 1.1:0.9 (or any value therebetween), and a statement that a four-way ratio is “about 5:3:1:1” can mean that the first number in the ratio can be any value of at least 4.5 and no more than 5.5, the second number in the ratio can be any value of at least 2.7 and no more than 3.3, and so on.

The use of “substantially” in the present disclosure, when referring to a measurable quantity (e.g., a diameter or other distance) and used for purposes of comparison, is intended to mean within 5% of the comparative quantity. The terms “substantially similar to,” “substantially the same as,” and “substantially equal to,” as used herein, should be interpreted as if explicitly reciting and encompassing the special case in which the items of comparison are “similar to,” “the same as” and “equal to,” respectively.

The term “a” or “an” entity, as used herein, refers to one or more of that entity. As such, the terms “a” (or “an”), “one or more” and “at least one” can be used interchangeably herein.

The use of “including,” “comprising,” or “having” and variations thereof herein is meant to encompass the items listed thereafter and equivalents thereof as well as additional items. Accordingly, the terms “including,” “comprising,” or “having” and variations thereof can be used interchangeably herein. The use of “engaged with” and variations thereof herein is meant to encompass any direct or indirect connections between components.

It shall be understood that the term “means” as used herein shall be given its broadest possible interpretation in accordance with 35 U.S.C. § 112 (f). Accordingly, a claim incorporating the term “means” shall cover all structures, materials, or acts set forth herein, and all of the equivalents thereof. Further, the structures, materials, or acts and the equivalents thereof shall include all those described in the summary, brief description of the drawings, detailed description, abstract, and claims themselves.

All external references are hereby incorporated by reference in their entirety whether explicitly stated or not.

These and other advantages will be apparent from the disclosure contained herein. The above-described embodiments, objectives, and configurations are neither complete nor exhaustive. The Summary is neither intended nor should it be construed as being representative of the full extent and scope of the present disclosure. Moreover, references made herein to “the present disclosure,” or aspects thereof should be understood to mean certain embodiments of the present disclosure and should not necessarily be construed as limiting all embodiments to a particular description. The present disclosure is set forth in various levels of detail in the Summary as well as in the attached drawings and the Detailed Description and no limitation as to the scope of the present disclosure is intended by either the inclusion or non-inclusion of elements, components, etc. in this Summary. Additional aspects of the present disclosure will become more readily apparent from the Detailed Description, particularly when taken together with the drawings.

It is to be appreciated that any embodiment, feature, or aspect described herein can be claimed in combination with any other embodiment(s), feature(s), or aspect(s) as described herein, regardless of whether the features or aspects come from the same described embodiment. For example, any one or more aspects described herein can be combined with any other one or more aspects described herein. In addition, any one or more features described herein can be combined with any other one or more features described herein. Further, any one or more embodiments described herein can be combined with any other one or more embodiments described herein.

### BRIEF DESCRIPTION OF THE DRAWINGS

Those of skill in the art will recognize that the following description is merely illustrative of the principles of the disclosure, which may be applied in various ways to provide many different alternative embodiments. This description is made for illustrating the general principles of the teachings of this disclosure and is not meant to limit the inventive concepts disclosed herein.

The accompanying drawings, which are incorporated in and constitute a part of the specification, illustrate embodiments and together with the general description of the disclosure given above and the detailed description of the drawings given below, serve to explain the principles of the disclosure. It is noted that any line in the drawings may be illustrated as solid or broken lines, including any section or length of each individual line, without departing from the scope of the present disclosure.

FIG. 1A illustrates a perspective view of a modular screw for a vertebral fixation system, in accordance with one or more embodiments of the present disclosure;

FIG. 1B illustrates a front elevation view of the modular screw of FIG. 1A;

FIG. 1C illustrates a side elevation view of the modular screw of FIG. 1A;

FIG. 1D illustrates a top plan view of the modular screw of FIG. 1A;

FIG. 2A illustrates a cross-section of a front elevation view of a portion of the modular screw of FIG. 1A with a first biasing member, in accordance with one or more embodiments of the present disclosure;

FIG. 2B illustrates a front elevation view of the portion of the modular screw with the first biasing member of FIG. 2A, where a housing of the modular screw is transparent;

FIG. 2C illustrates a side elevation view of the portion of the modular screw with the first biasing member of FIG. 2A, where the housing of the modular screw is transparent;

FIG. 2D illustrates a top plan view of the portion of the modular screw with the first biasing member of FIG. 2A, where the housing of the modular screw is transparent;

FIG. 2E illustrates an exploded view of a portion of a variation of the modular screw with the first biasing member of FIG. 2A, in accordance with one or more embodiments of the present disclosure;

FIG. 2F illustrates a front elevation view of the portion of the variation of the modular screw with the first biasing member of FIG. 2E;

FIG. 3A illustrates a cross-section of a front elevation view of a portion of a variation of the modular screw of FIG. 2A with the first biasing member, in accordance with one or more embodiments of the present disclosure;

FIG. 3B illustrates a front elevation view of the portion of the variation of the modular screw with the first biasing member of FIG. 3A, where a housing of the modular screw is transparent;

FIG. 3C illustrates a side elevation view of the portion of the variation of the modular screw with the first biasing member of FIG. 3A, where the housing of the modular screw is transparent;

FIG. 3D illustrates an exploded view of the portion of the variation of the modular screw with the first biasing member of FIG. 3A;

FIG. 4A illustrates a perspective view of a portion of a second variation of the modular screw of FIG. 2A with the first biasing member, where a housing of the modular screw is transparent, in accordance with one or more embodiments of the present disclosure;

FIG. 4B illustrates a front elevation view of the portion of the second variation of the modular screw with the first biasing member of FIG. 4A, where the housing of the modular screw is transparent;

FIG. 4C illustrates a side elevation view of the portion of the second variation of the modular screw with the first biasing member of FIG. 4A, where the housing of the modular screw is transparent;

FIG. 4D illustrates a top plan view of the portion of the second variation of the modular screw with the first biasing member of FIG. 4A, where the housing of the modular screw is transparent;

FIG. 4E illustrates an exploded view of the portion of the second variation of the modular screw with the first biasing member of FIG. 4A;

FIG. 5 illustrates a cross-section of a front elevation view of a portion of the modular screw of FIG. 1A with a second biasing member, in accordance with one or more embodiments of the present disclosure;

FIG. 6A illustrates a cross-section of a front elevation view of a portion of the modular screw of FIG. 1A with a third biasing member, in accordance with one or more embodiments of the present disclosure;

FIG. 6B illustrates a front elevation view of the portion of the modular screw with the third biasing member of FIG. 6A, where a housing of the modular screw is transparent;

FIG. 6C illustrates a side elevation view of the portion of the modular screw with the first biasing member of FIG. 6A, where the housing of the modular screw is transparent;

FIG. 6D illustrates a top plan view of the portion of the modular screw with the first biasing member of FIG. 6A, where the housing of the modular screw is transparent;

FIG. 6E illustrates an exploded view of the portion of the modular screw with the first biasing member of FIG. 6A;

FIG. 7 illustrates a flow diagram of a method or process for the operation and use of a vertebral fixation system including the modular screw of FIGS. 1A-6E, in accordance with one or more embodiments of the present disclosure;

FIG. 8A illustrates a cross-section of a perspective view of a portion of a second variation of the modular screw of FIG. 1A, where the second variation of the modular screw is at least partially pre-assembled prior to insertion, in accordance with one or more embodiments of the present disclosure;

FIG. 8B illustrates the cross-section of the perspective view of the portion of the second variation of the modular screw of FIG. 8A;

FIG. 9 illustrates a flow diagram of a method or process for the operation and use of a vertebral fixation system including the modular screw of FIGS. 8A and 8B, in accordance with one or more embodiments of the present disclosure; and

FIG. 10 illustrates a front elevation view of a portion of a third variation of the modular screw of FIG. 1A, in accordance with one or more embodiments of the present disclosure.

It should be understood that the drawings are not necessarily to scale, and various dimensions may be altered. In certain instances, details that are not necessary for an understanding of the disclosure or that render other details difficult to perceive may have been omitted. It should be understood, of course, that the disclosure is not necessarily limited to the particular embodiments illustrated herein. It is noted that any line in the drawings may be illustrated as solid or broken lines, including any section or length of each individual line, without departing from the scope of the present disclosure.

| Reference Number | Component |
|---|---|
| 100 | Modular Screw |
| 102 | Fastener |
| 104 | Shank |
| 106 | Head |
| 108 | Housing |
| 110 | Base |
| 112 | Arm |
| 114 | Channel |
| 116 | Head Cavity |
| 118 | Head Aperture |
| 120 | Tool Engagement Feature |
| 122 | Spring Cavity |
| 124 | Piston |
| 126 | Piston Cavity |
| 128 | Distal Tip |
| 130 | Tool Engagement Feature |
| 132 | Interlocking Mechanism |
| 134 | Housing Assembly |
| 136 | Ring |
| 200 | Biasing Member |
| 202 | Pin |
| 204 | Pin Spring |
| 206 | Spring End Cavity |
| 208 | Protrusion |
| 210 | Groove |
| 212 | Piston Surface |
| 214 | Head Surface |
| 216 | Protrusion |
| 218 | Groove |
| 220 | Exterior Surface |
| 222 | Interior Surface |
| 224 | First Portion of Piston |
| 226 | Second Portion of Piston |
| 228 | Trunnion |
| 300 | Ring |
| 302 | Ring Cavity |
| 400 | Locking Assembly |
| 402 | Member Channel |
| 404 | Member |
| 406 | Pin |
| 408 | Pin Cavity |
| 410 | End |
| 412 | End |
| 500 | Biasing Member |
| 502 | Spring |
| 504 | Spring End Cavity |
| 600 | Biasing Member |
| 602 | Integrally Formed Spring |
| 604 | Housing Spring Cavity |
| 606 | Spring Cavity Surface |
| 608 | Washer |
| 610 | Groove |
| 700 | Method or Process |
| 702 | Provide Fastener of Modular Screw |
| 704 | Couple Housing to Fastener |
| 706 | Actuate Housing to Cause Fastener to Engage Piston |
| 708 | Engage Head of Fastener with Ring |
| 710 | Apply a Force to Locking Assembly |
| 712 | Insert Stabilization Member and/or |
| | Set Screw into Housing |
| 800 | First Portion of Head of Fastener |
| 802 | Second Portion of Head of Fastener |
| 804 | Pin Aperture in Housing |
| 806 | Pin |
| 808 | First Portion of Pin |
| 810 | Second Portion of Pin |
| 812 | Shear Point |
| 814 | Pin Aperture in Head of Fastener |
| 816 | Exterior Surface of Piston |
| 818 | Interior Surface of Housing |
| 820 | Pass-Through Aperture |
| 822 | Tool Pin |
| 824 | Pin Aperture in Piston |
| 826 | Pre-Assembly or Housing Assembly |
| 830 | Set Screw |
| 900 | Method or Process |
| 902 | Provide Pre-Assembly with Housing |
| | of Modular Screw |
| 904 | Couple Pre-assembly to Fastener |
| | of Modular Screw |
| 906 | Modify Pre-Assembly for Polyaxial Movement |
| 908 | Insert Stabilization Member and/or |
| | Set Screw into Housing |
| 1000 | Shank Bore |
| 1002 | Head Shaft |
| 1004 | Interlocking Assembly |
| 1006 | Contoured Surface of Shank |
| 1008 | Contoured Surface of Head |
| 1010 | Stabilization Member |

### DETAILED DESCRIPTION

Although the following text sets forth a detailed description of numerous different embodiments, it should be understood that the legal scope of the description is defined by the words of the claims set forth at the end of this disclosure. The Detailed Description is to be construed as exemplary only and does not describe every possible embodiment since describing every possible embodiment of the modular screw would be impractical, if not impossible. Numerous alternative embodiments could be implemented, using either current technology or technology developed after the filing date of this patent, which would still fall within the scope of the claims. Additionally, any combination of features shown in the various figures can be used to create additional embodiments of the present disclosure. Thus, dimensions, aspects, and features of one embodiment of the modular screw can be combined with dimensions, aspects, and features of another embodiment of the modular screw to create the claimed embodiment.

In general, embodiments of the present disclosure are directed to a modular screw for a vertebral fixation system, and a method or process regarding the installation or operation of the same. The modular screw includes a fastener and a housing. A head of the fastener is insertable into the housing via a bottom-loading procedure, where the fastener is first inserted into a vertebra and the distal end of the housing is then coupled to the head of the fastener. Coupling of the housing to the head of the fastener causes a biased piston (i.e., by biasing members) inserted within the housing to transition toward a proximal end of the housing, releasing a ring that held in place between the piston and the housing. Release of the ring causes the ring to transition into a position where the ring engages the head of the fastener, preventing the head of the fastener from exiting the distal end of the housing.

Embodiments of the present disclosure are also directed to the biasing members including a spring (or combination of spring and pin) being inserted into the housing (i.e., through the sidewalls of the housing) and engaging the piston. Engagement with the piston by the biasing members causes the biasing of the piston within the housing, toward the distal end of the housing, absent a force being applied to the piston (i.e., by the head of the fastener). Embodiments of the present disclosure are also directed to the spring being integrally formed with the piston. Further, embodiments of the present disclosure are directed to the piston being a single-piece component or a multi-piece component or assembly.

Embodiments of the present disclosure are further directed to a pre-assembled housing assembly including the housing, the piston, a second portion of the head of the fastener that is able to receive a first portion of the head of the fastener, and a pin that secures the second portion of the head of the fastener to the housing. The pin prevents polyaxial movement of the housing about the head of the fastener, but can be sheared or otherwise broken with the application of a pre-determined amount of force to allow for polyaxial movement of the housing about the head of the fastener. Embodiments of the present disclosure are further directed to reduce or preventing polyaxial movement of the piston relative to the housing, including by contact between interior housing and exterior piston surfaces that may be tapered and/or via contact by an installation tool to tool pins that pass through the housing and are coupled to the piston.

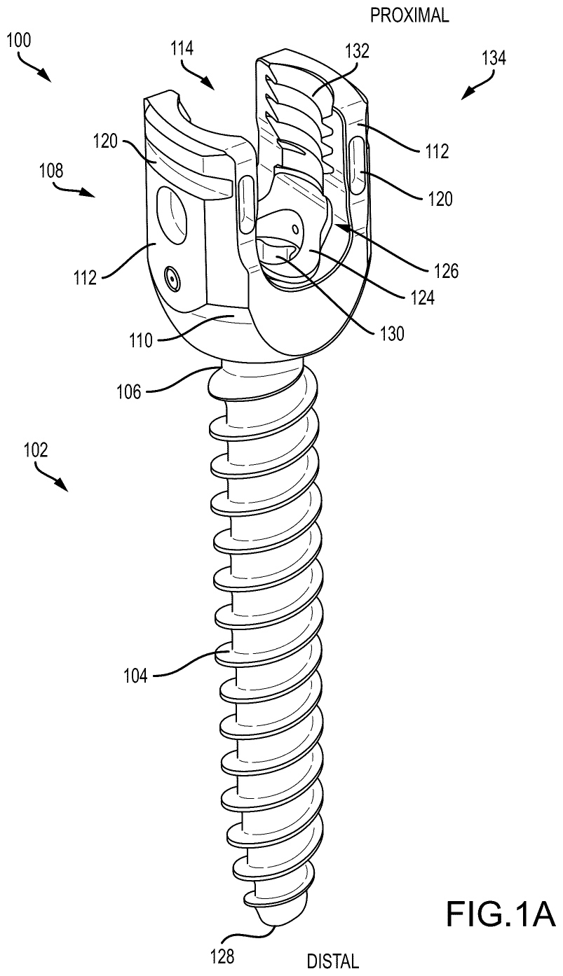

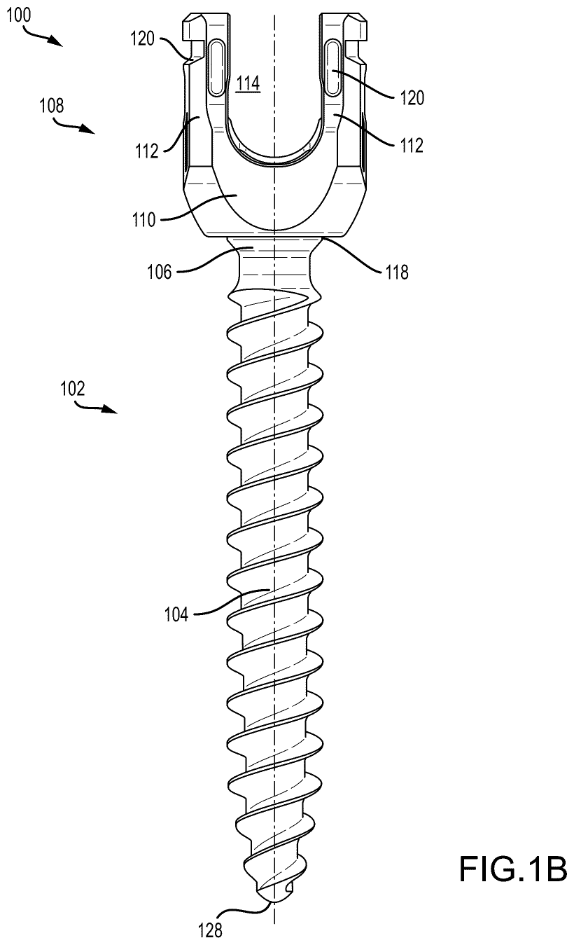

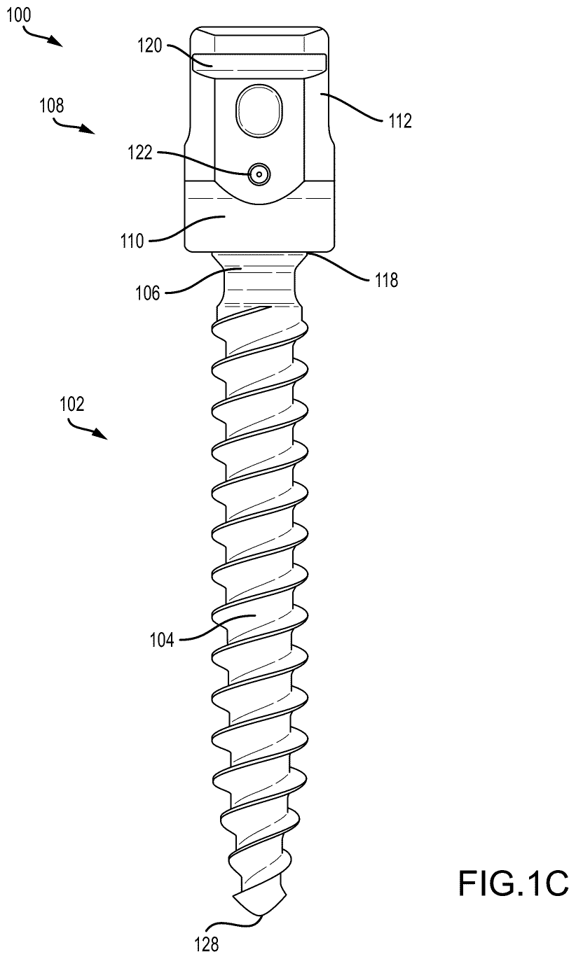

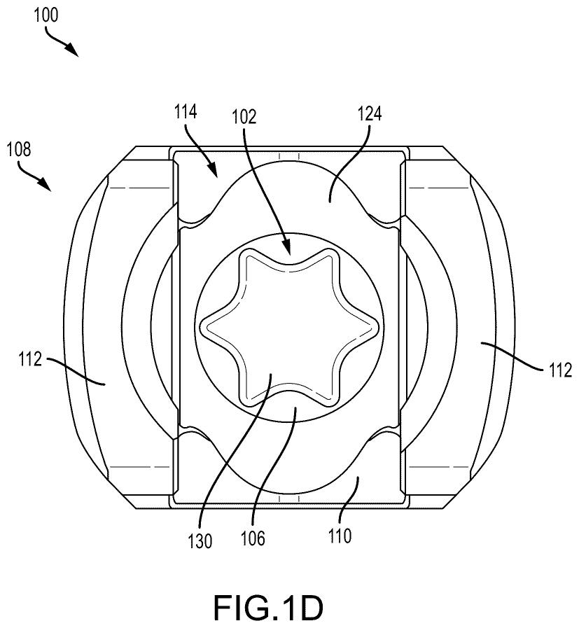

FIGS. 1A-1D in general illustrate a modular screw 100, in accordance with one or more embodiments of the present disclosure. The modular screw 100 includes a fastener 102 with a shank 104 and a head 106.

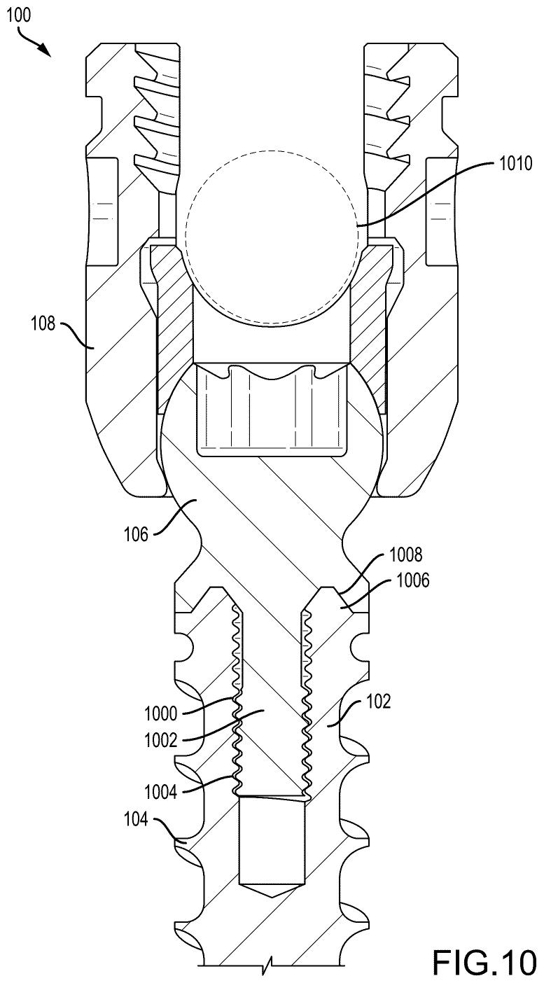

The modular screw 100 includes a housing 108 with a base 110 and at least one arm 112 that extends from the base 110. The at least one arm 112 defines a channel 114, into which a stabilization member (e.g., stabilization member 1010, as illustrated in FIG. 10) may be inserted. For example, the stabilization member may be a flexible or rigid member such as a cord, a rod, or the like.

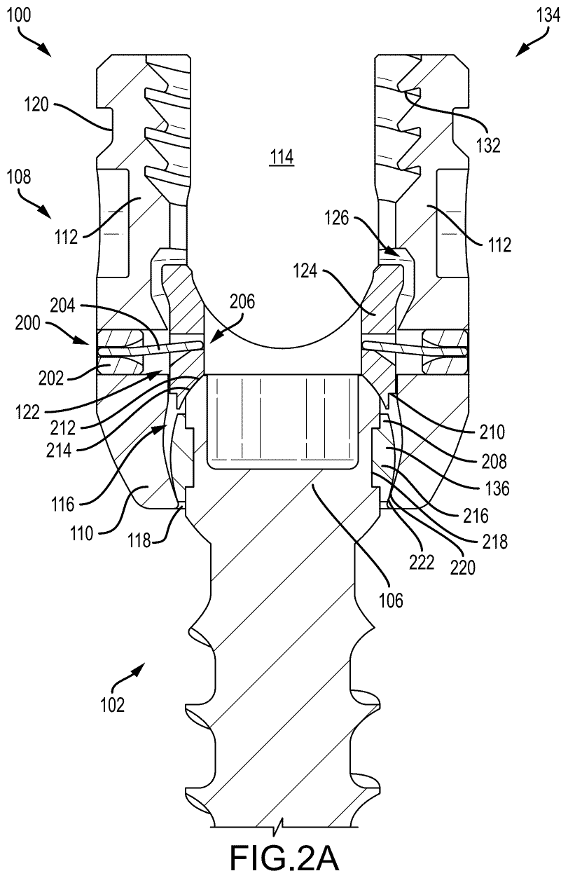

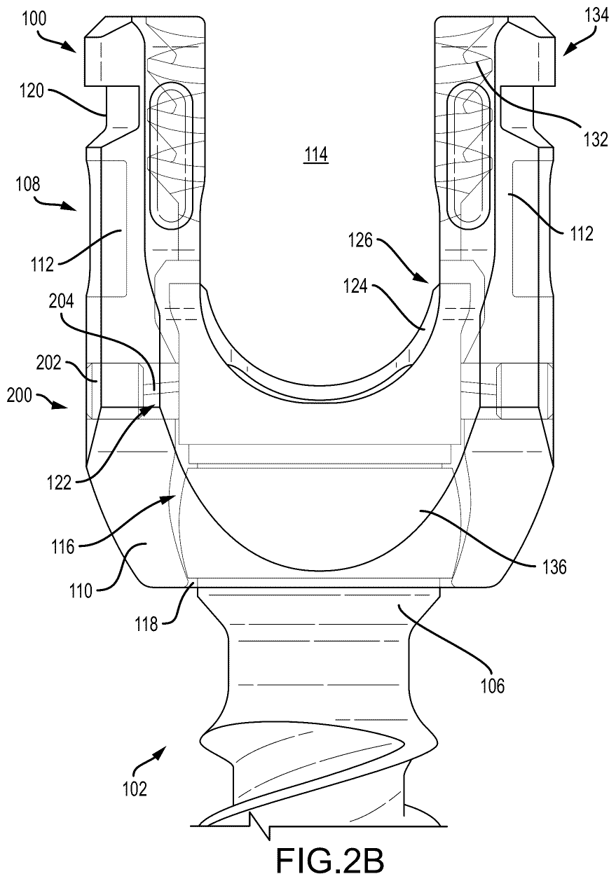

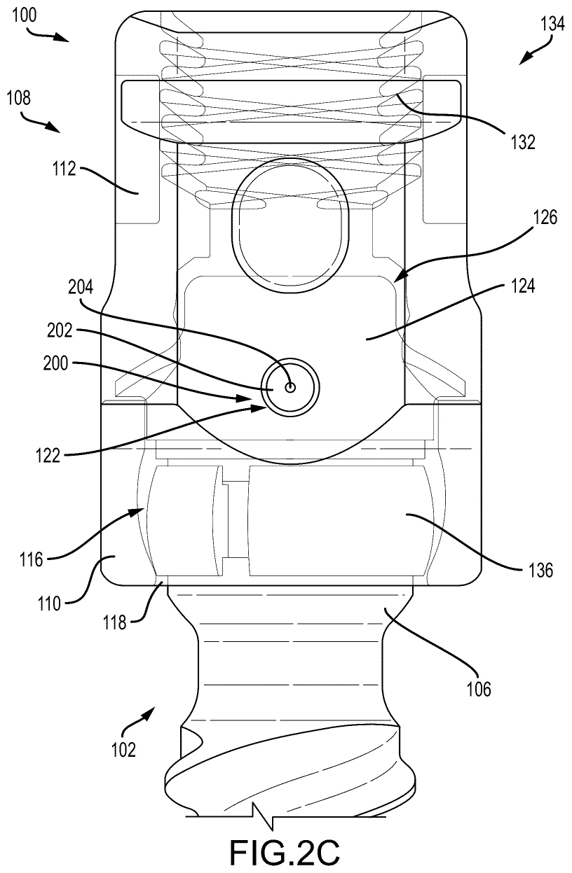

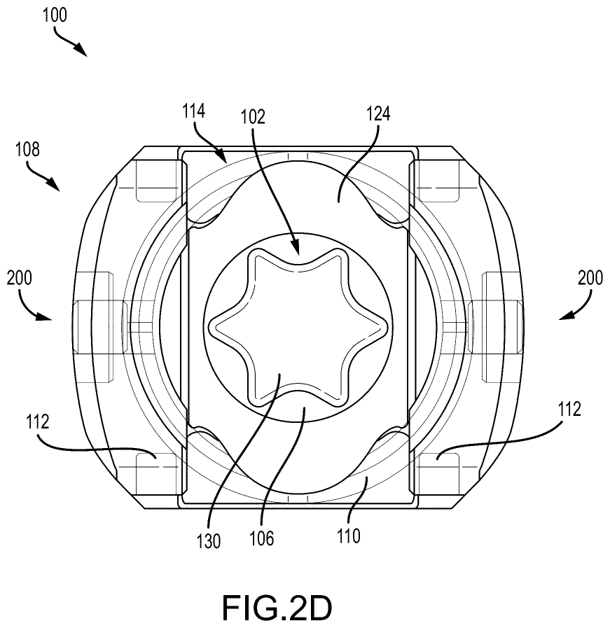

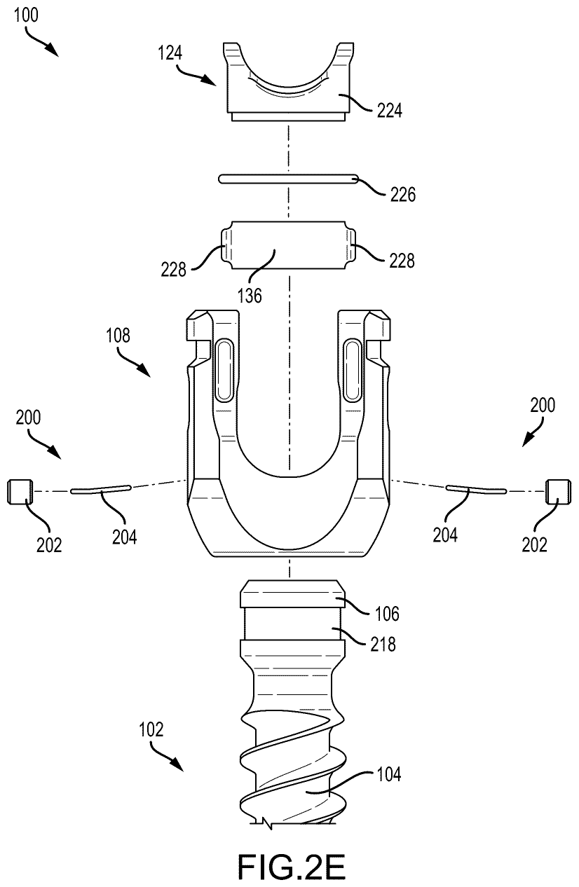

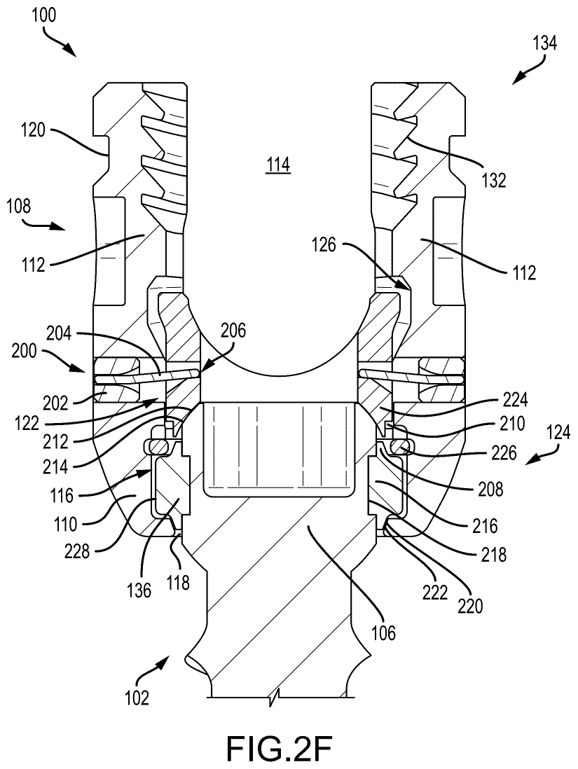

The housing 108 further includes a head cavity 116 (e.g., as illustrated in at least FIG. 2A). The head 106 of the fastener 102 is insertable into the head cavity 116 via a head aperture 118 at a distal end of the housing 108. For example, the head 106 is bottom loaded into the housing 108 through the head aperture 118 and into the head cavity 116. It is noted that the head cavity 116 may be a portion of the channel 114, or may be defined within the housing 108 and separate from the channel 114, without departing from the scope of the present disclosure.

In some embodiments, the housing 108 includes one or more tool engagement features 120. For example, the tool engagement features 120 is connectable to a tool that assists in installing the housing 108 on the head 106 of the fastener 102. As discussed in detail further herein, the tool may be used to position the housing 108 on the head 106, and further to actuate (i.e., depress) the housing 108 to attach to the head 106. It is noted, however, that the housing 108 may be installed on the fastener 102 without need for a tool, such that the one or more tool engagement features 120 are optional, without departing from the scope of the present disclosure.

The housing 108 includes one or more spring cavities 122. Each of the one or more spring cavities 122 is configured to receive one or more biasing members 200, 500, 600. As discussed in detail further herein, the one or more biasing members 200, 500, 600 are able to move a piston 124 inserted in a piston cavity 126 (e.g., as illustrated in at least FIG. 2A) within the housing 108, causing the piston 124 to engage and seat against the head 106 of the fastener 102.

In embodiments, the fastener 102 includes a distal tip 128 of the shank 104 and a tool engagement feature 130 at a proximal end of the head 106. The tool engagement feature 130 is accessible through the housing 108 (and, in some embodiments, through the piston 124) via the channel 114. The tool engagement feature 130 may include any standard or proprietary pattern known in the art, including a star or Torx® pattern, a cross-head or Phillips-head pattern, a slot pattern, or other pattern known in the art.

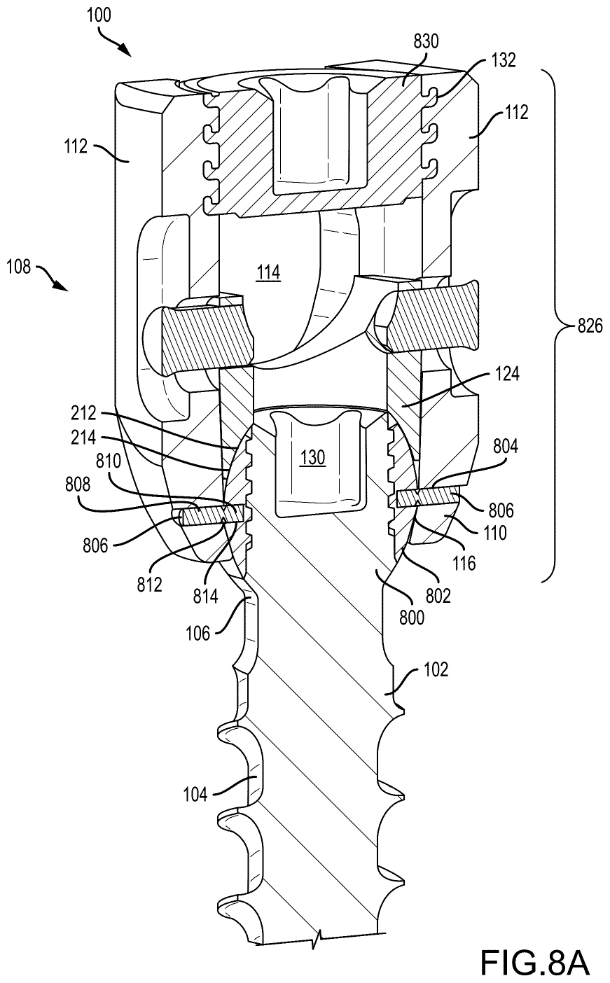

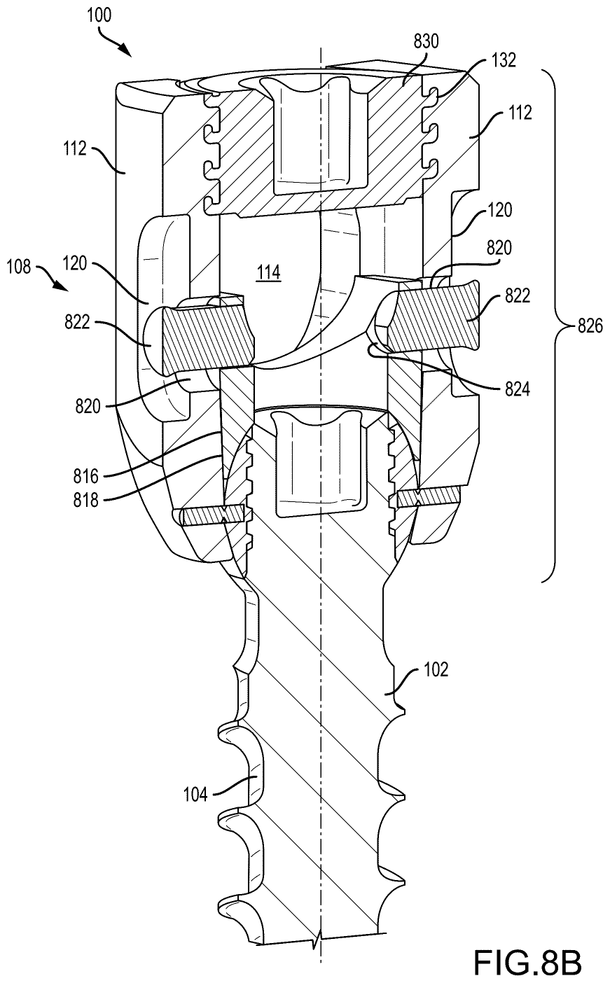

In embodiments, the housing 108 includes an interlocking mechanism 132, for engaging with a component to hold the stabilization member within the channel 114. For example, the interlocking mechanism 132 may include threading that is complementary to threading of a set screw (e.g., a set screw 830, as illustrated in FIGS. 8A and 8B). By way of another example, the interlocking mechanism 132 may be a protrusion and/or groove able to engage with a corresponding groove and/or protrusion on a component that fits within the channel 114. It is noted that engaging the housing 108 with the component via the interlocking mechanism 132 may cause the component to engage with (e.g., push against) the stabilization member, causing the stabilization member to engage with (e.g., push against) the piston 124 within the piston cavity 126, thus securing the stabilization member within the channel 114 when the component is tightened or otherwise engaged. In addition, it is noted that engaging the housing 108 with the component via the interlocking mechanism 132 may cause the component to engage with (e.g., push against) the stabilization member, causing the stabilization member to engage with (e.g., push against) the piston 124 within the piston cavity 126 and causing the piston 124 to engage with (e.g., push against) the head 106 of the fastener 102, thus securing the piston 124 against the fastener 102. It is contemplated that sufficient pressure by the piston 124 on the head 106 may prevent polyaxial movement (or unidirectional movement) of the head 106 of the fastener 102 relative to the housing 108.

In embodiments, the modular screw 100 includes a housing assembly 134. For example, the housing assembly 134 may include, but is not limited to, the housing 108, the piston 124, and one or more of the biasing members 200, 500, 600 (e.g., as illustrated in FIGS. 2A-2F, 3A-3D, 4A-4E, 5, and/or 6A-6E), and as described in detail further herein. In addition, the housing assembly 134 may include a ring 136 (e.g., as illustrated in FIGS. 2A-2F, 3A-3D, 4A, 4E, 5, and/or 6A-6E), and as described in detail further herein.

As part of the modularity of the screw 100, the housing assembly 134 may be fully built prior to coupling of the housing 108 to the head 106 of the fastener 102. Alternatively, some or all of the housing assembly 134 may be installed within the housing 108 after the housing 108 is coupled to the head 106, without departing from the scope of the present disclosure.

FIGS. 2A-2F in general illustrate the modular screw 100 with the one or more biasing members 200, in accordance with one or more embodiments of the present disclosure. It should be understood that embodiments or aspects directed to the modular screw 100 of FIGS. 1A-1D may be directed to the modular screw 100 as illustrated in FIGS. 2A-2F with the one or more biasing members 200 unless specifically noted, and vice versa, without departing from the scope of the present disclosure.

The one or more biasing members 200 each include a pin 202 that houses a first end of a pin spring 204. A second end of the pin spring 204 is insertable into a spring end cavity 206 within the piston 124 (e.g., within an exterior sidewall of the piston 124). It is noted that the cavities 122, 206 may be considered components of the biasing member 200 without departing from the scope of the present disclosure.

In embodiments, the one or more biasing members 200 cause the piston 124 to bias (i.e., toward the distal end of the housing 108) within the piston cavity 126 of the housing 108, as the second end of the pin spring 204 is in contact with the spring end cavity 206 of the piston 124. While the piston 124 is initially biased and prior to insertion of the head 106 of the fastener 102 into the housing 108, a protrusion 208 of the ring 136 is caught within a groove 210 defined by the exterior surface of the piston 124 and the interior surface of the housing 108 that forms the piston cavity 126. Being caught within the groove 210, the protrusion 208 holds the ring 136 in a proximal position within the head cavity 116. It is noted that the piston cavity 126 may be a portion of the channel 114, or may be defined within the housing 108 and separate from the channel 114, without departing from the scope of the present disclosure. In addition, it is noted that the piston 124 may at least partially extend into the head cavity 116.

The housing 108 is then coupled to the head 106, via insertion of the head 106 into the head aperture 118 and the head cavity 116 of the housing 108 in a bottom-loading direction (i.e., from the distal end and toward the proximal end of the housing 108). While the ring 136 is in the proximal position, the head 106 is able to enter the head cavity 116 and engage a distal end of the piston 124. For example, the distal end of the piston 124 may include a piston surface 212 that is contoured to conform a head surface 214.

A continued application of force on the distal end of the piston 124 by the head 106 causes the piston 124 to transition toward the proximal end of the housing 108, overcoming the biasing force applied on the piston 124 by the one or more biasing members 200. Beyond a certain threshold or height of the piston 124 within the piston cavity 126, the groove 210 no longer engages the protrusion 208 of the ring 136.

The contoured surfaces within the head cavity 116 (and gravity) causes the protrusion 208 of the ring 136 to disengage from the groove 210 of the piston 124, allowing the ring 136 to transition toward the distal end of the housing 108. During and/or following the movement of the ring 136 toward the distal end of the housing 108, a second protrusion 216 of the ring 136 engages with a second groove 218 within the head 106 of the fastener 102. It is noted that the head 106 is able to be removed from the head cavity 116 prior to the coupling of the ring 136 to the head 106 via the engagement of the second protrusion 216 and the second groove 218, but is held in place within the head cavity 116 after the coupling of the ring 136 to the head 106 via the engagement of the second protrusion 216 and the second groove 218 due to the interference between an exterior surface 220 of the ring 136 and an interior surface 222 of the housing 108 that defines the head cavity 116. In addition, it is noted that FIG. 2A specifically illustrates the coupling of the ring 136 to the head 106 via the respective engaging second protrusion 216 and second groove 218.

Although the ring 136 is described as being held in a proximal position, it is noted that the ring 136 may transition to a further proximal position toward the proximal end of the housing 108 a select amount with the piston 124 while the application of force by the head 106 on the piston 124 occurs. In other embodiments, the ring 136 may be held by the piston 124 at the proximal-most position (e.g., with the contoured surfaces within the head cavity 116 preventing actuation of the ring 136 in the proximal direction) via the biasing force applied by the one or more biasing members 200, until actuation of the piston 124 by the engagement of the head 106 on the piston 124 to counter the biasing force applied by the one or more biasing members 200 causes the piston 124 to release the ring 136.

Although embodiments of the present disclosure illustrated in FIGS. 2A-2D include a single-piece piston 124, it is noted that the piston 124 may be a multi-piece component. For example, as illustrated in FIGS. 2E and 2F, the piston 124 may include a first portion 224 and a second portion 226. In this example, the first portion 224 includes the spring end cavity 206. In addition, the second portion 226 includes the groove 210 operable to engage the protrusion 208 of the ring 136. Further, the first portion 224 includes the piston surface 212 that is contoured to conform to the head surface 214. The multi-piece piston 124, and in particular the second portion 226 of the piston 124, provides additional flexibility for the polyaxial movement of the housing 108 about the head 106 of the fastener 102. In particular, the flexibility of the second portion 226 may assist in the locking and/or unlocking of the polyaxial movement of the housing 108 about the head 106 of the fastener 102.

As illustrated in FIGS. 2E and 2F, in some embodiments the ring 136 includes one or more trunnions 228. For example, the trunnions 228 may be horizontally opposed boss features on the ring 136. In some instances, the trunnions 228 are cylindrical features on the ring 136.

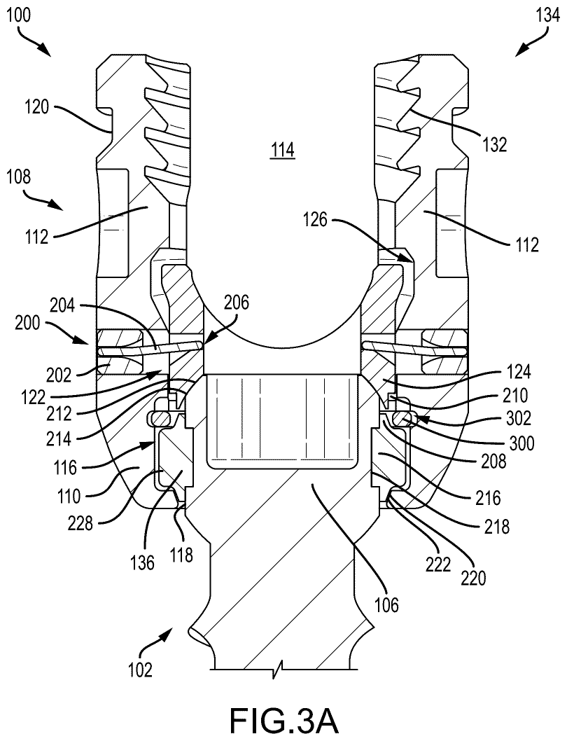

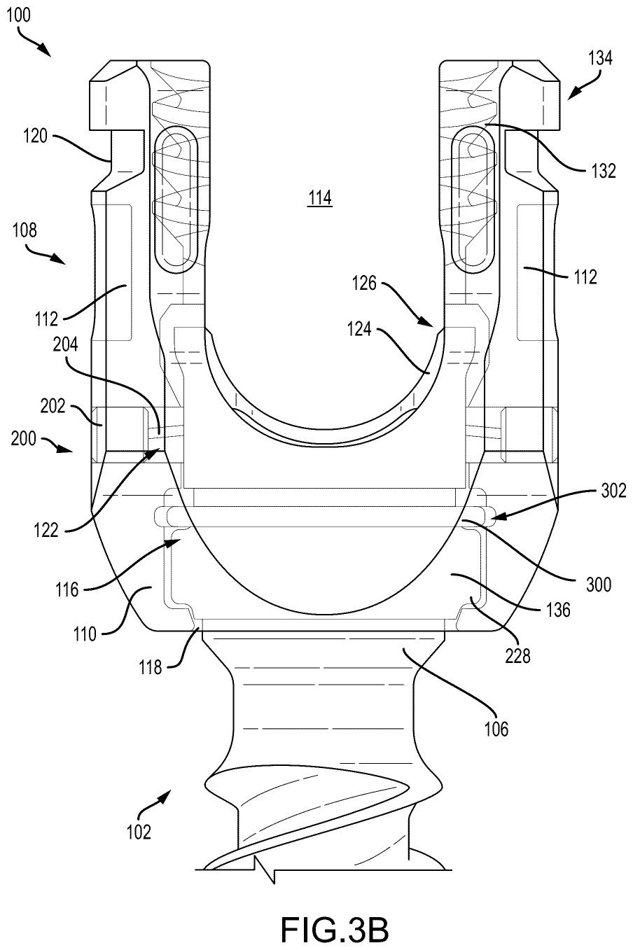

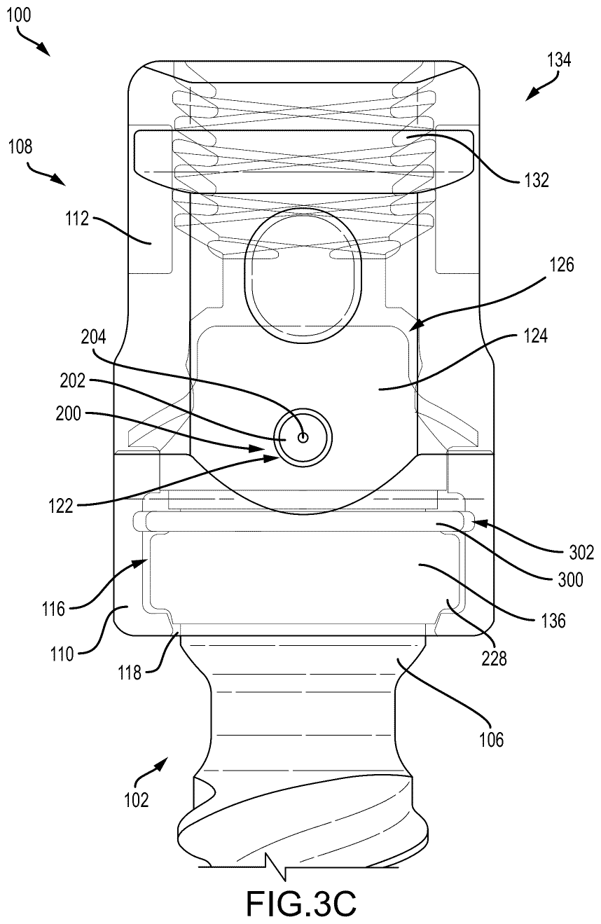

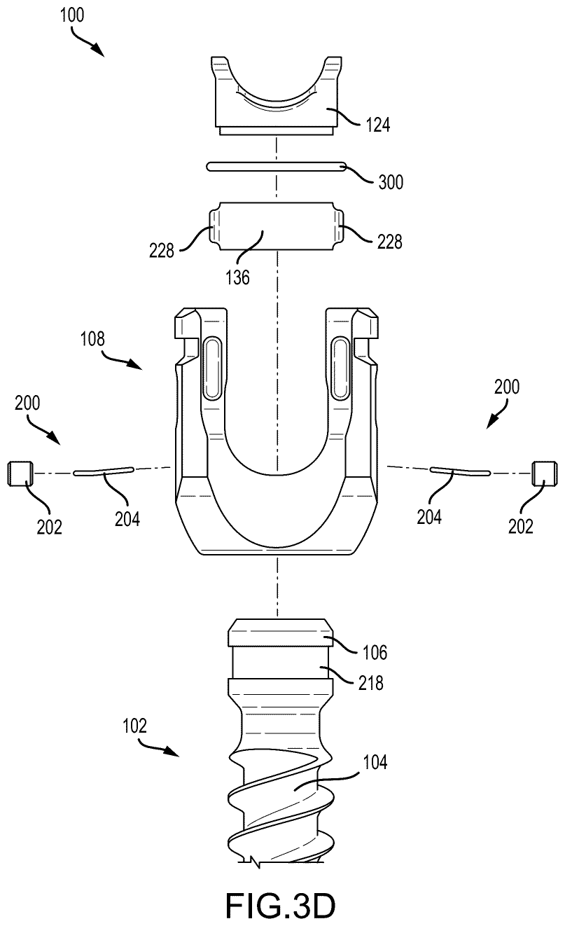

FIGS. 3A-3D in general illustrate the modular screw 100 with the one or more biasing members 200, in accordance with one or more embodiments of the present disclosure. In particular, FIGS. 3A-3D illustrate a variation of the modular screw 100 in FIGS. 2A-2F, in which the modular screw 100 is configured for unidirectional movement of the head 106 of the fastener 102 relative to the housing 108. It should be understood that embodiments or aspects directed to the modular screw 100 of FIGS. 1A-1D and 2A-2F may be directed to the modular screw 100 as illustrated in FIGS. 3A-3D with the one or more biasing members 200 unless specifically noted, and vice versa, without departing from the scope of the present disclosure.

In FIGS. 3A-3D, the modular screw 100 is limited to or unidirectional movement of the head 106 of the fastener 102 relative to the housing 108 via a ring 300. It is noted that the ring 300 may be the same component as the second portion 226 of the piston 124, or may be another component within the modular screw 100 in addition to (or instead of) the second portion 226 of the piston 124, without departing from the scope of the present disclosure. As such, in some embodiments, it should be understood that features illustrated in FIGS. 2E-2F with respect to the piston 124 including the first portion 224 and the second portion 226 may read on the ring 300, and vice versa, without departing from the scope of the present disclosure.

The ring 300 is positioned within a ring cavity 302 defined in the housing 108. It is noted that the ring cavity 302 may be a portion of the channel 114, the head cavity 116, and/or the piston cavity 126. Alternatively, the ring cavity 302 may be a separate cavity defined within the housing 108 without departing from the scope of the present disclosure.

The ring 300 is operable as a blocker to prevent polyaxial movement (or reduce movement to only unidirectional movement) of the head 106 of the fastener 102 relative to the housing 108. For example, engagement of one or more of the housing 108, the piston 124, the ring 136, the ring 300, and/or the head 106 of the fastener 102 prevents polyaxial movement (or reduces movement to only unidirectional movement) of the head 106 of the fastener 102 relative to the housing 108.

In particular, the ring 300 expands into the ring cavity 302, which allows the ring 136 to expand and be assembled onto the groove 210 within the housing 108 in a preloaded condition (i.e., or an “as shipped” condition). When the shank 104 of the fastener 102 is loaded, which pushes the head 106 of the fastener 102 into the piston 124 and actuates the piston 124 into a more proximal position within the housing 108, the ring 136 separates from the piston 124 and releases the potential energy of the ring 136 in the expanded state. Release of the ring 136 allows the ring 136 to collapse around the head 106 of the fastener 102, capturing the head 106 of the fastener 102 in the housing 108.

The force supplied by the biasing member 200 additionally actuates the piston 124 to a more distal position within the housing 108, allowing the head 106 of the fastener 102 and the now-collapsed (i.e., and locked) ring 136 to drop below the ring 300. The ring 300 then collapses above the trunnions 228 (i.e., as illustrated in FIG. 3A), retaining the head 106 of the fastener 102 and the ring 136 (i.e., as an assembly) in the more distal position within the housing 108. It is noted that when a medial lateral force is placed on the housing 108, the housing 108 and the ring 300 will provide opposite forces on the trunnions 228, thus eliminating polyaxial movement in an axial direction of the trunnions 228.

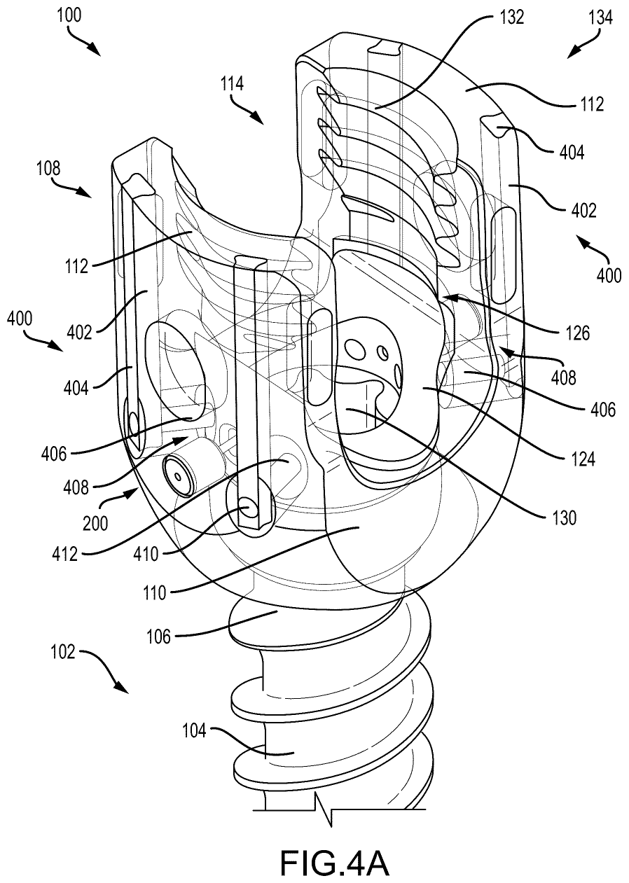

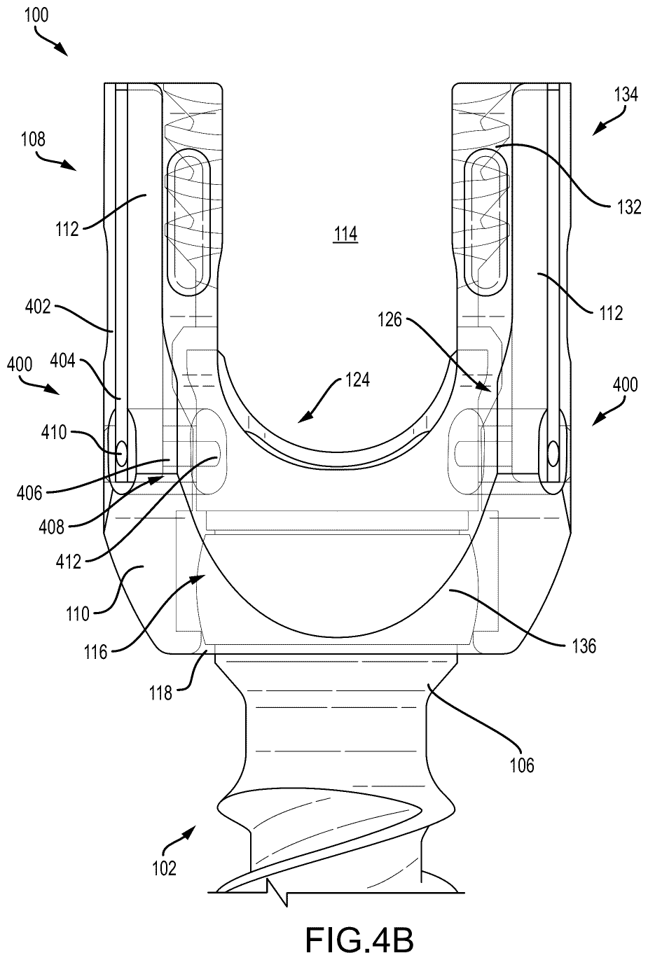

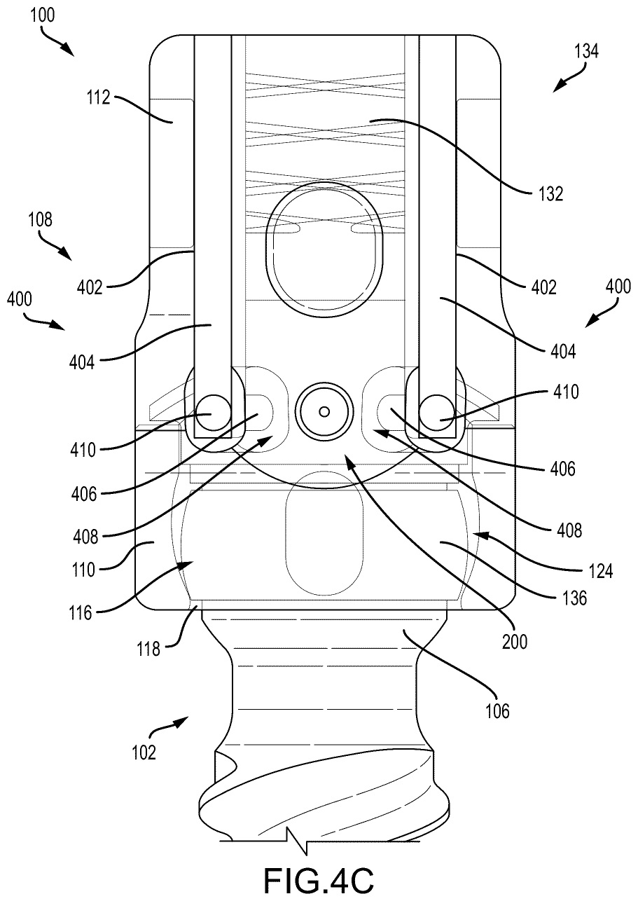

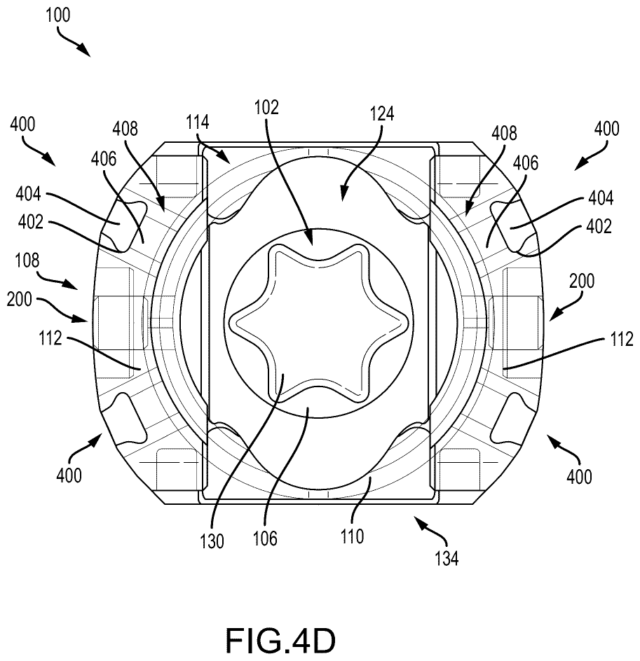

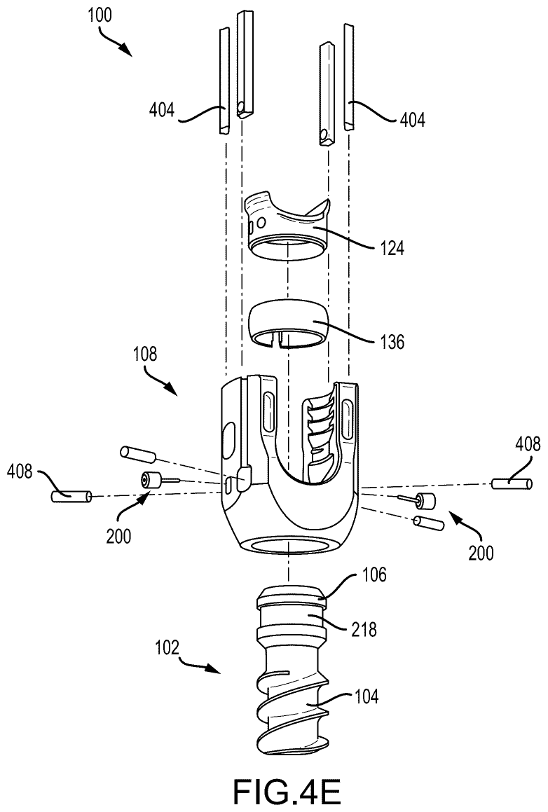

FIGS. 4A-4E in general illustrate the modular screw 100 with the one or more biasing members 200, in accordance with one or more embodiments of the present disclosure. In particular, FIGS. 4A-4E illustrate a second variation of the modular screw 100 in FIGS. 2A-2F, in which the modular screw 100 is configured for locking and unlocking the housing assembly 134 to the head 106 of the fastener 102 via a locking assembly 400. It should be understood that embodiments or aspects directed to the modular screw 100 of FIGS. 1A-1D, 2A-2F, and 3A-3D may be directed to the modular screw 100 as illustrated in FIGS. 4A-4E with the one or more biasing members 200 unless specifically noted, and vice versa, without departing from the scope of the present disclosure.

In FIGS. 4A-4E, the housing 108 of the modular screw 100 includes one or more member channels 402, that are operable to receive respective members 404. For example, the one or more member channels 402 may run longitudinally along an exterior of the housing 108, or between the proximal and distal ends of the housing 108.

The housing 108 includes one or more pins 406 within respective pin cavities 408 through the housing 108. The pins 406 have a first end 410 that is configured to engage with respective members 404, and a second end 412 that is configured to engage with the piston 124 (i.e., an exterior surface of the piston 124 or an exterior surface of the first portion 224 of the piston 124, where the piston 124 is a multi-piece component).

An axial force or pressure applied to a particular member 404 (i.e., from an instrument or from another component of the modular screw 100, not illustrated herein) is loaded onto the first end 410 of the corresponding radial pin 406. The force is transferred through the pin 406 and to the piston 124 via the second end 412 of the pin 406.

It is noted that the ability for polyaxial or monoaxial rotation is available depending on which of the members 404 and pins 406 are locked via the force (i.e., axial force) being applied to the members 404 (i.e., which prevents movement of the piston 124 and thus the head 106 of the fastener 102 relative to the housing 108). In addition, it is noted that the housing 108 may be completely locked and unable to rotate about the head 106 of the fastener 102 if a sufficient force is applied to all members 404 and pins 406.

It is noted that stabilization members and/or interlocking components (i.e., that engage with the interlocking mechanism 132 of the housing 108) may be installed within the channel 114 without unlocking the piston 124 through removal of the force (i.e., axial force) applied to the members 404 (and thus the pins 406).

Although not shown, it should be understood that the housing 108 may include the tool engagement features 120 in addition to the member channels 404 without departing from the scope of the present disclosure. In addition, it should be understood that the housing 108 may include the ring cavity 302 for the ring 300, and/or the first portion 224 and the second portion 226 of the piston 124 with the trunnions 228 of the ring 136, in addition to the locking assembly 400 without departing from the scope of the present disclosure.

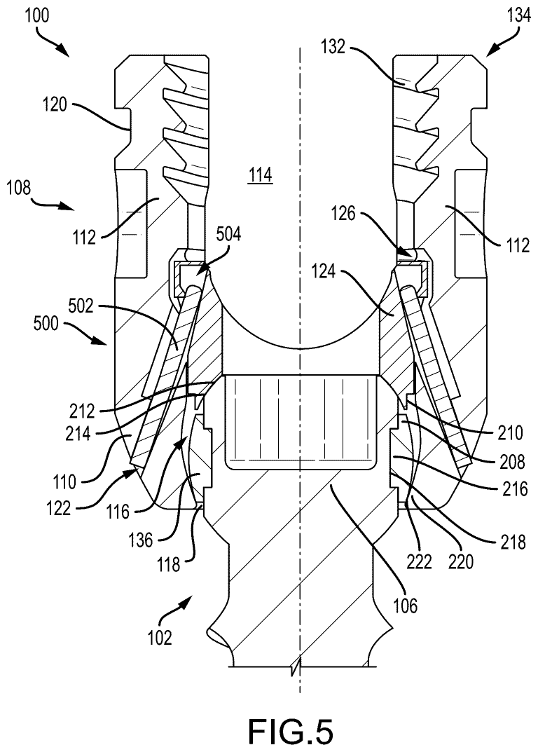

FIG. 5 illustrates the modular screw 100 with the one or more biasing members 500, in accordance with one or more embodiments of the present disclosure. It should be understood that embodiments or aspects directed to the modular screw 100 of FIGS. 1A-1D may be directed to the modular screw 100 as illustrated in FIG. 5 with the one or more biasing members 500 unless specifically noted, and vice versa, without departing from the scope of the present disclosure. In addition, it should be understood that one or more components of the one or more biasing members 200 and one or more components of the one or more biasing members 500 may be combinable or interchangeable, without departing from the scope of the present disclosure. Further, it should be understood that the housing 108 may include the ring cavity 302 for the ring 300, and/or the first portion 224 and the second portion 226 of the piston 124 with the trunnions 228 of the ring 136, in addition to the biasing members 500 without departing from the scope of the present disclosure. Further, it should be understood that the biasing members 500 and the locking assembly 400 may be combined within the housing 108 to provide a locking feature to the modular screw 100 with the biasing members 500 without departing from the scope of the present disclosure.

The one or more biasing members 500 each include a spring 502, where a first end of the spring 502 is in the spring cavity 122 of the housing 108. A second end of the spring 502 is insertable into a spring end cavity 504 within the piston 124 (e.g., within an exterior sidewall of the piston 124). It is noted that the cavities 122, 504 may be considered components of the one or more biasing members 500 without departing from the scope of the present disclosure.

In embodiments, the one or more biasing members 500 cause the piston 124 to bias (i.e., toward the distal end of the housing 108) within the piston cavity 126 of the housing 108, as the second end of the spring 502 is in contact with the spring end cavity 504 of the piston 124. While the piston 124 is initially biased and prior to insertion of the head 106 of the fastener 102 into the housing 108, the protrusion 208 of the ring 136 is caught within the groove 210 defined by the exterior surface of the piston 124 and the interior surface of the housing 108 that forms the piston cavity 126. Being caught within the groove 210, the protrusion 208 holds the ring 136 in a proximal position within the head cavity 116.

It should be understood that the insertion angle of the spring 502 within the spring cavity 122 and the spring end cavity 504 is not limited to the illustrated embodiment in FIG. 5. Rather, the spring cavity 122 may be formed in the housing 108 at any angle that still allows for the biasing of the piston 124 (e.g., at an angle that is substantially perpendicular to a longitudinal axis through the housing 108, at an angle that is substantially parallel to the longitudinal axis through the housing 108, or any oblique angle therebetween).

The housing 108 is then coupled to the head 106, via insertion of the head 106 into the head aperture 118 and the head cavity 116 of the housing 108 in a bottom-loading direction (i.e., from the distal end and toward the proximal end of the housing 108). While the ring 136 is in the proximal position, the head 106 is able to enter the head cavity 116 and engage a distal end of the piston 124. For example, the distal end of the piston 124 may include the piston surface 212 that is contoured to conform the head surface 214.

A continued application of force on the distal end of the piston 124 by the head 106 causes the piston 124 to transition toward the proximal end of the housing 108, overcoming the biasing force applied on the piston 124 by the one or more biasing members 500. Beyond a certain threshold or height of the piston 124 within the piston cavity 126, the groove 210 no longer engages the protrusion 208 of the ring 136. It is noted that FIG. 5 specifically illustrates the contact of head surface 214 and the piston surface 212, and post-release of the protrusion 208 of the ring 136 from the groove 210 of the piston 124.

The contoured surfaces within the head cavity 116 (and gravity) causes protrusion 208 of the ring 136 to disengage from the groove 210 of the piston 124, allowing the 136 to transition toward the distal end of the housing 108. During and/or following the movement of the ring 136 toward the distal end of the housing 108, the second protrusion 216 of the ring 136 engages with the second groove 218 within the head 106 of the fastener 102. It is noted that the head 106 is able to be removed from the head cavity 116 prior to the coupling of the ring 136 to the head 106 via the engagement of the second protrusion 216 and the second groove 218, but is held in place within the head cavity 116 after the coupling of the ring 136 to the head 106 via the engagement of the second protrusion 216 and the second groove 218 due to the interference between an exterior surface 220 of the ring 136 and the interior surface 222 of the housing 108 that defines the head cavity 116.

Although the ring 136 is described as being held in a proximal position, it is noted that the ring 136 may transition to a further proximal position toward the proximal end of the housing 108 a select amount with the piston 124 while the application of force by the head 106 on the piston 124 occurs. In other embodiments, the ring 136 may be held by the piston 124 at the proximal-most position (e.g., with the contoured surfaces within the head cavity 116 preventing actuation of the ring 136 in the proximal direction) via the biasing force applied by the one or more biasing members 500, until actuation of the piston 124 by the engagement of the head 106 on the piston 124 to counter the biasing force applied by the one or more biasing members 500 causes the piston 124 to release the ring 136.

Although embodiments of the present disclosure illustrated in FIG. 5 include a single-piece piston 124, it is noted that the piston 124 may be a multi-piece component. For example, the piston 124 may be a multi-piece component including the first portion 224 and the second portion 226, as illustrated in FIGS. 2E and 2F, without departing from the scope of the present disclosure. For example, the first portion 224 may include the spring end cavity 504 that is configured to receive the second end of the spring 502.

In embodiments, the ring 136 illustrated in FIGS. 2A-2F, 3A-3D, 4A-4E, and 5 may be non-continuous in shape, with at least one separation line or point through the height of the ring 136 that allows for the expansion and contraction of the ring 136 between a first diameter (i.e., when the second protrusion 216 enters the second groove 218 of the head 106) and a second diameter (i.e., when the protrusion 208 is in the groove 210). For example, the ring 136 may include, but is not limited to, a c-ring. It is noted herein, however, that the ring 136 may be a single loop without departing from the scope of the present disclosure. In general, the ring 136 may be fabricated from a medical-grade biocompatible material that has a sufficient elasticity to allow for deformation from a first diameter to a second diameter, and return from the second diameter to the first diameter.

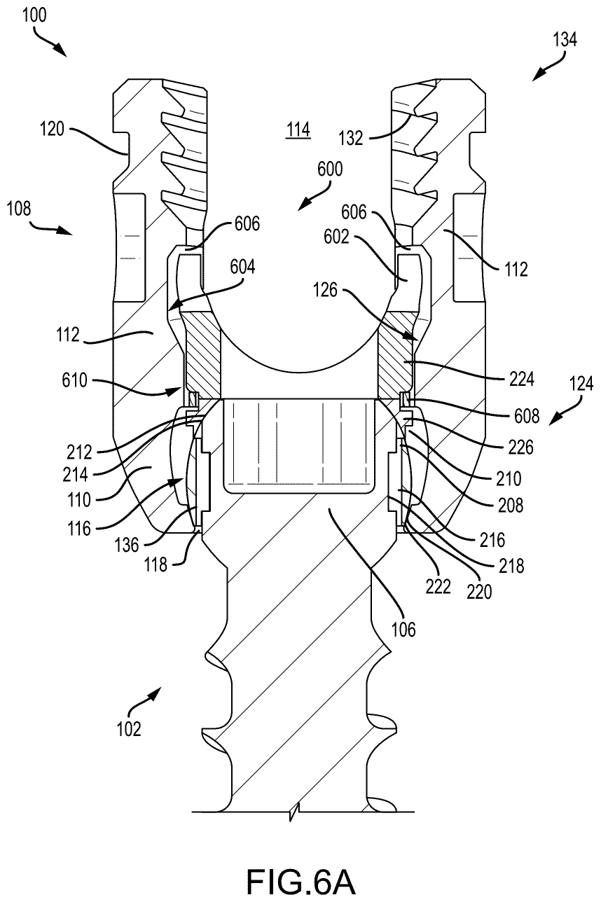

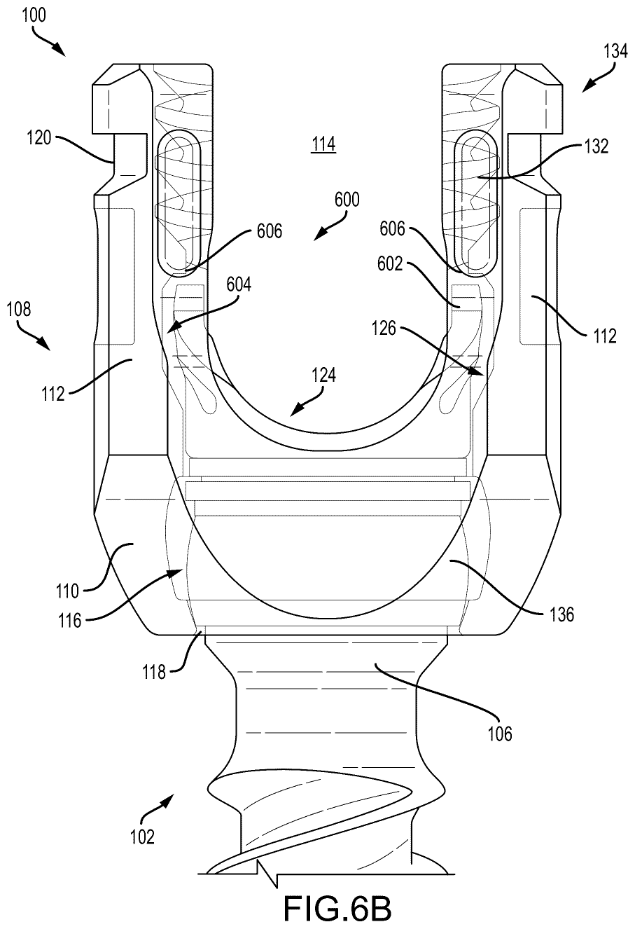

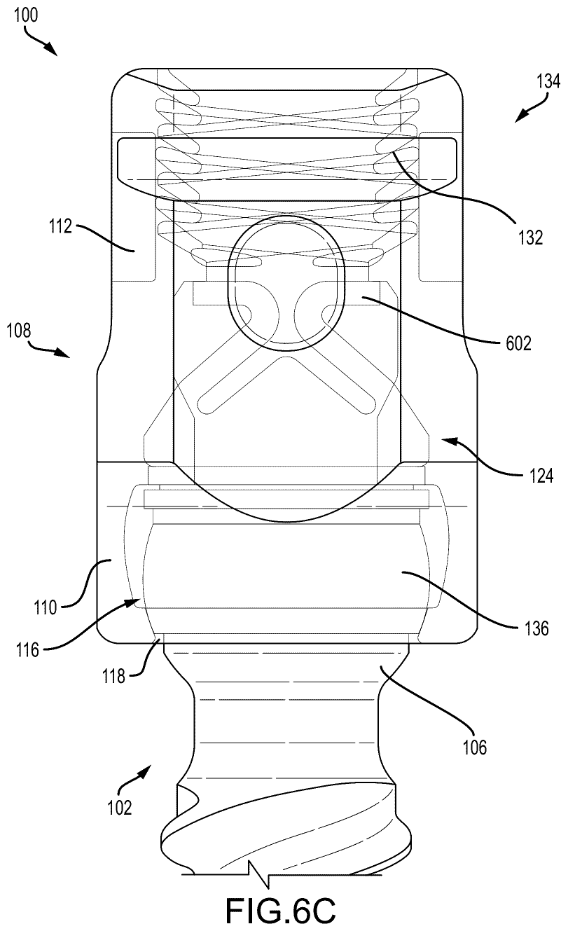

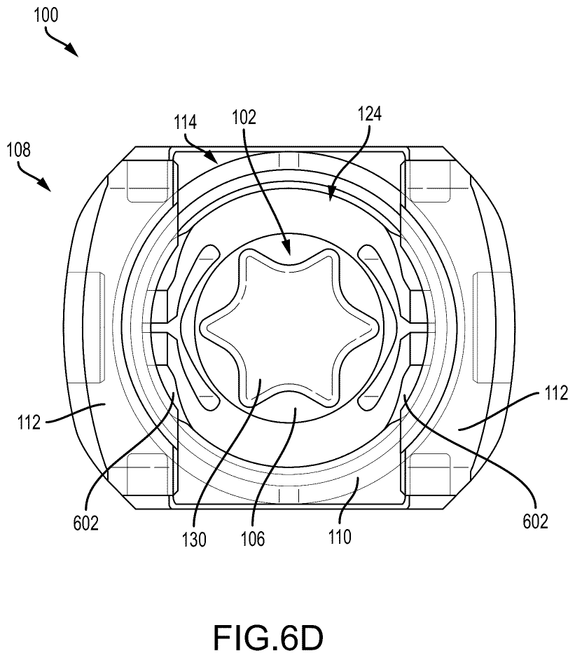

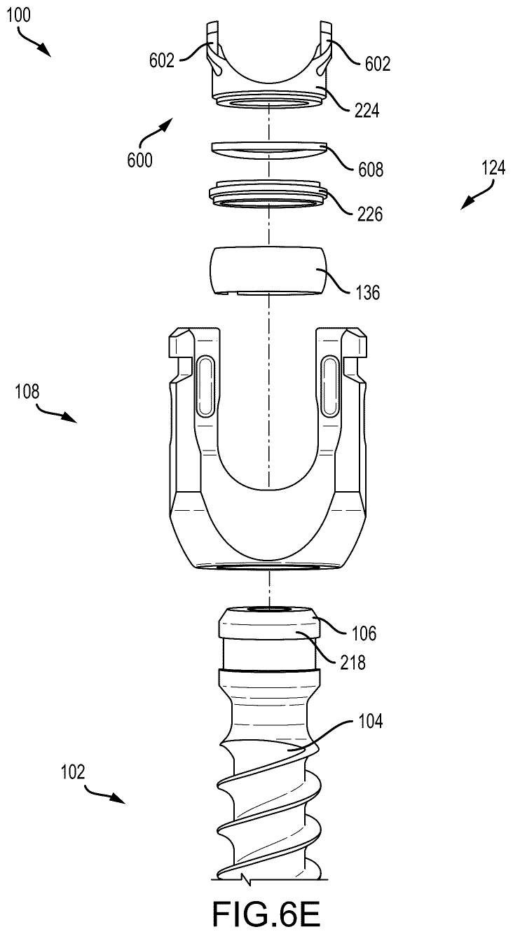

FIGS. 6A-6E in general illustrate the modular screw 100 with the one or more biasing members 600, in accordance with one or more embodiments of the present disclosure. It should be understood that embodiments or aspects directed to the modular screw 100 of FIGS. 1A-1D may be directed to the modular screw 100 as illustrated in FIGS. 6A-6E with the one or more biasing members 600 unless specifically noted, and vice versa, without departing from the scope of the present disclosure. In addition, it should be understood that one or more components of the one or more biasing members 200, one or more components of the one or more biasing members 500, and/or one or more components of the one or more biasing members 600 may be combinable or interchangeable without departing from the scope of the present disclosure. Further, it should be understood that the housing 108 may include the ring cavity 302 for the ring 300, and/or the first portion 224 and the second portion 226 of the piston 124 with the trunnions 228 of the ring 136, in addition to the biasing members 600 without departing from the scope of the present disclosure. Further, it should be understood that the biasing members 600 and the locking assembly 400 may be combined within the housing 108 to provide a locking feature to the modular screw 100 with the biasing members 600 without departing from the scope of the present disclosure.

The one or more biasing members 600 each include an integrally formed spring 602. For example, the integrally formed spring 602 may be formed with the piston 124. By way of another example, the integrally formed spring 602 may be coupled to the piston 124. The integrally formed spring 602 is positioned within a housing spring cavity 604. It is noted that the housing spring cavity 604 may be a portion of the piston cavity 126, or may be a separate cavity defined within the housing 108, without departing from the scope of the present disclosure. It is noted that the housing spring cavity 604 may be considered a component of the biasing member 600 without departing from the scope of the present disclosure.

In embodiments, the one or more biasing members 600 cause the piston 124 to bias (i.e., toward the distal end of the housing 108) within the piston cavity 126 of the housing 108, as the integrally formed spring 602 is in contact with a spring cavity surface 606 of the housing spring cavity 604 defined within the housing 108. While the piston 124 is initially biased and prior to insertion of the head 106 of the fastener 102 into the housing 108, the protrusion 208 of the ring 136 is caught within the groove 210 defined by the exterior surface of the piston and the interior surface of the housing 108 that forms the piston cavity 126. Being caught within the groove 210, the protrusion 208 holds the ring 136 in a proximal position within the head cavity 116.

The housing 108 is then coupled to the head 106, via insertion of the head 106 into the head aperture 118 and the head cavity 116 of the housing 108 in a bottom-loading direction (i.e., from the distal end and toward the proximal end of the housing 108). While the ring 136 is in the proximal position, the head 106 is able to enter the head cavity 116 and engage a distal end of the piston 124. For example, the distal end of the piston 124 may include the piston surface 212 that is contoured to conform the head surface 214.

A continued application of force on the distal end of the piston 124 by the head 106 causes the piston 124 to transition toward the proximal end of the housing 108, further compressing the integrally formed springs 602 against the spring cavity surfaces 606. Beyond a certain threshold or height of the piston 124 within the piston cavity 126, the groove 210 no longer engages the protrusion 208 of the ring 136.

The contoured surfaces within the head cavity 116 (and gravity) causes the protrusion 208 of the ring 136 to disengage from the groove 210 of the piston 124, allowing the ring 135 to transition toward the distal end of the housing 108. During and/or following the movement of the ring 136 toward the distal end of the housing 108, the second protrusion 216 of the ring 136 engages with the second groove 218 within the head 106 of the fastener 102. It is noted that the head 106 is able to be removed from the head cavity 116 prior to the coupling of the ring 136 to the head 106 via the engagement of the second protrusion 216 and the second groove 218, but is held in place within the head cavity 116 after the coupling of the ring 136 to the head 106 via the engagement of the second protrusion 216 and the second groove 218 due to the interference between an exterior surface 220 of the ring 136 and the interior surface 222 of the housing 108 that defines the head cavity 116. In addition, it is noted that FIG. 6A specifically illustrates the coupling of the ring 136 to the head 106 via the respective engaging second protrusion 216 and second groove 218.

Although the ring 136 is described as being held in a proximal position, it is noted that the ring 136 may transition to a further proximal position toward the proximal end of the housing 108 a select amount with the piston 124 while the application of force by the head 106 on the piston 124 occurs. In other embodiments, the ring 136 may be held by the piston 124 at the proximal-most position (e.g., with the contoured surfaces within the head cavity 116 preventing actuation of the ring 136 in the proximal direction) via the biasing force applied by the one or more biasing members 600, until actuation of the piston 124 by the engagement of the head 106 on the piston 124 to counter the biasing force applied by the one or more biasing members 600 causes the piston 124 to release the ring 136.

Although embodiments of the present disclosure illustrated in FIGS. 6A-6E include a single-piece piston 124, it is noted that the piston 124 may be a multi-piece component. For example, the piston 124 may include the first portion 224 and the second portion 226. In this example, the first portion 224 includes the integrally formed spring 602. In addition, the second portion 226 includes the groove 210 operable to engage the protrusion 208 of the ring 136. Further, the second portion 226 includes the piston surface 212 that is contoured to conform to the head surface 214.

In some embodiments, the modular screw 100 includes a washer 608. For example, where the piston 124 includes the first portion 224 and the second portion 226, the washer 608 may be positioned within a groove 610 formed between the first portion 224 and the second portion 226. It is noted that the washer 608 may be a wave spring in some embodiments. In addition, it is noted that the washer 608 may be considered a standalone component that engages the piston 124, or may be a component that is part of an assembly including the piston 124 (and/or components of the piston 124 such as the first portion 224 and the second portion 226), without departing from the scope of the present disclosure.

It is noted that embodiments of the present disclosure illustrate a polyaxial design, where the piston 124 is contoured to allow for polyaxial movement of the piston 124 and the housing 108 relative to the head 106 of the fastener 102. However, it should be understood that the piston 124 may be designed and/or the housing assembly 134 may include additional components that limit the head 106 of the fastener 102 to unidirectional movement relative to the housing 108 (or housing assembly 134) without departing from the scope of the present disclosure.

It is noted that one or a plurality of modular screws 100 may be considered a component of a vertebral fixation system including one or more stabilization members, one or more components such as set screws, and the like, for purposes of the present disclosure. In addition, it is noted that the vertebral fixation system may include and/or be configured to be acted upon by one or more tools (i.e., one or more minimally invasive surgical tools, and the like).

Although embodiments of the present disclosure illustrated in FIGS. 2A-2F, 3A-3D, 4A-4E, 5, and 6A-6E are directed to the insertion of the head 106 into the housing 108, it should be understood that the present disclosure is directed to the fastener 102 already being installed within a vertebra of a patient or other holding device prior to the insertion of the head 106 into the housing 108. As such, the housing 108 is configured to receive the head 106 when the housing 108 is coupled to the head 106. An application of force on the housing 108 causes the head 106 to be inserted into the housing 108, and eventual re-positioning and engaging of the ring 136 within the head cavity 116 to the head 106.

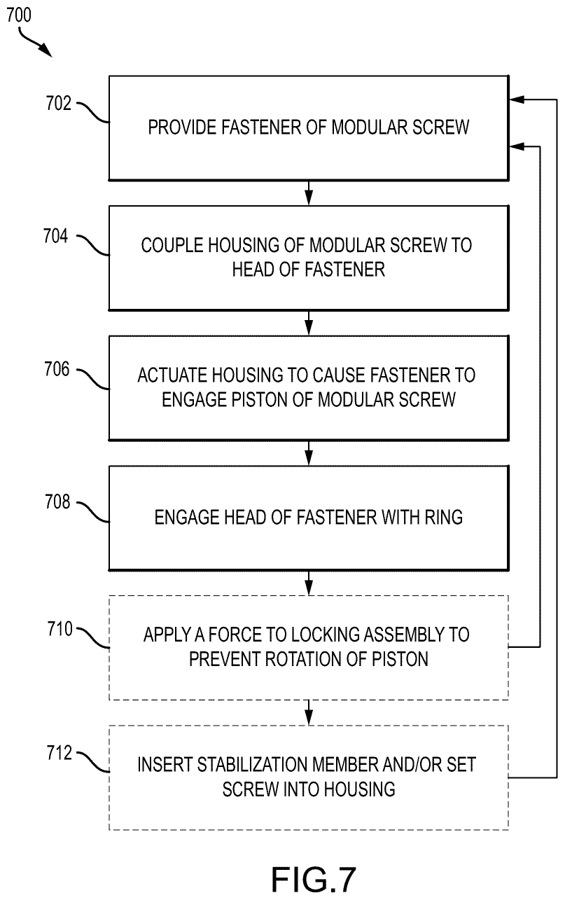

FIG. 7 is a method or process flow diagram 700 illustrating the installation and operation of a vertebral fixation system including the modular screw 100, in accordance with one or more embodiments or the present disclosure. While a general order for the operations of the method or process is shown in FIG. 7, the method or process can include more or fewer operations or can arrange the order of the operations differently (including simultaneously, substantially simultaneously, or sequentially) than those shown in FIG. 7. It is noted that the method or process shall be explained with reference to the components, devices, subassemblies, environments, etc. described in conjunction with FIGS. 1A-6E. For example, it is noted that the embodiments as illustrated in FIGS. 1A-6E should be understood as reading on the embodiments described with respect to FIG. 7, and vice versa, without departing from the scope of the present disclosure.

In embodiments, a fastener of a modular screw is provided 702. In some embodiments, the distal tip 128 of the shank 104 of the fastener 102 is inserted into the vertebrae of the patient or other holding device. It is noted that one or more pre-processing procedures may occur to the vertebrae (i.e., drilling, etc.) and/or the fastener 102 prior to insertion.

In embodiments, a housing of the modular screw is coupled 704 to the head of the fastener. In embodiments, the head 106 passes through a head aperture 118 and into a head cavity 116 of the housing 108. A head surface 214 makes contact with a piston surface 212 of a piston 124 inserted within a piston cavity 126 defined within the housing 108. In embodiments, one or more biasing members 200, 500, 600 cause the piston 124 to bias (i.e., toward the distal end of the housing 108) within a piston cavity 126 of the housing 108. While a ring 136 is in the proximal position within the housing 108, the head 106 is able to enter the head cavity 116 and engage a distal end of the piston 124. For example, the distal end of the piston 124 may include a piston surface 212 that is contoured to conform a head surface 214.

In embodiments, the housing is actuated 706 to cause the fastener to transition the piston of the modular screw. A continued application of force on the distal end of the piston 124 by the head 106 resulting from the actuation (i.e., depressing) of the housing 108 causes the piston 124 to transition toward the proximal end of the housing 108. Beyond a certain threshold or height of the piston 124 within the piston cavity 126, the groove 210 no longer engages the protrusion 208 of the ring 136.

In embodiments, a ring engages 708 the head of the fastener. The contoured surfaces within the head cavity 116 (and gravity) causes the ring 136 to transition toward the distal end of the housing 108. Following the movement of the ring 136, a second protrusion 216 of the ring 136 engages with (or seats within) a second groove 218 within the head 106 of the fastener 102. It is noted that the head 106 is able to be removed from the head cavity 116 prior to the coupling of the ring 136 to the head 106, but is held in place within the head cavity 116 after the coupling of the ring 136 to the head 106 due to the interference between an exterior surface 220 of the ring 136 and the interior surface 222 of the housing 108 that defines the head cavity 116.

In embodiments, a force is applied 710 to a locking assembly to prevent rotation of the piston. In embodiments where the modular screw 100 includes the locking assembly 400, a force is applied to one or more of the members 404, which is transferred via the corresponding pins 406 to the piston 124. Engagement of the piston 124 with the second end 412 of the pin 406 prevents rotation in a select direction of the head 106 of the fastener 102 relative to the housing 108. It is noted that the application 710 of the force may be optional, such as where rotation of the piston 124 is not desired.

In embodiments, a stabilization member and/or a set screw is inserted 712 into the housing. In embodiments, the stabilization member (e.g., stabilization member 1010, as illustrated in FIG. 10) is inserted into the channel 114, and optionally at least partially contacts the piston 124. In addition (or in the alternative), the set screw (e.g., a set screw 830, as illustrated in FIGS. 8A and 8B) or other interlocking component is inserted into the channel 114, where the set screw or other interlocking component is configured to engage the interlocking mechanism 132 of the housing 108. For example, the engaging the interlocking mechanism 132 may secure the set screw or other interlocking component within the housing 108 to hold the stabilization member within the channel 114. It is noted that the insertion of the stabilization member and/or the set screw is optional, such as where additional modular screws 100 are installed in additional vertebrae prior to insertion of the stabilization member and/or the set screw in the implanted modular screw 100, to allow for the positioning and securing of multiple modular screws 100 with the stabilization member and/or respective set screws together.

In embodiments, one or more of the providing 702, coupling 704, actuation 706, engagement 708, application of force 710, and insertion 712 may be performed for implantation of additional modular screws 100 in additional vertebrae.

Although embodiments of the method or process 700 may include individual insertion of components of the modular screw 100 into a patient, it is noted that the vertebral fixation system including the modular screw 100 may be combined using at least operations 702, 704, 706, 708, 710, 712 exterior to and without contact with the human body. For example, the components may be combined during testing and/or experimentation, clinical studies and/or training seminars for how to use the vertebral fixation system including the modular screw 100, or another scenario in which the vertebral fixation system is not yet coupled to a patient but instead where the fastener 102 is inserted into a holding device (e.g., hard surface, ballistic gel, artificial or removed osseous structure, or the like) that mimics the installation surface of a vertebra.

Although above embodiments of the present disclosure include individual assembly of components of the modular screw 100, it is noted that the modular screw 100 may at least partially pre-assembled (i.e., prior to insertion into a patient, or into a holding device).

FIGS. 8A and 8B illustrate the modular screw 100 that is at least partially assembled (e.g., pre-assembled) prior to insertion, in accordance with one or more embodiments of the present disclosure. It should be understood that embodiments or aspects directed to the modular screw 100 of FIGS. 1A-1D, 2A-2F, 3A-3D, 4A-4E, 5, and/or 6A-6E may be directed to the pre-assembled components of the modular screw 100 as illustrated in FIGS. 8A and 8B unless specifically noted, and vice versa, without departing from the scope of the present disclosure. In addition, it should be understood that the housing 108 may include one or more components of the one or more biasing members 200, the ring cavity 302 for the ring 300, the first portion 224 and the second portion 226 of the piston 124 with the trunnions 228 of the ring 136, the locking assembly 400, one or more components of the one or more biasing members 500, and/or one or more components of the one or more biasing members 600, in addition to the pre-assembled components of the modular screw 100 in FIGS. 8A and 8B without departing from the scope of the present disclosure.

In embodiments, the modular screw 100 includes the fastener 102 with the shank 104 and the head 106. However, as illustrated in FIGS. 8A and 8B, the head 106 includes a first portion 800 and a second portion 802, where the first portion 800 engages the second portion 802 (e.g., are threadably engaged, includes corresponding components of an interlocking assembly, or the like).

In embodiments, the housing 108 includes the base 110 and the at least one arm 112 that extends from the base 110. The channel 114 is defined by the at least one arm 112. The housing 108 additionally includes the interlocking mechanism 132 for a set screw 830 or other interlocking component.

The housing 108 further includes the head cavity 116 accessible via the head aperture 118 at the distal end of the housing 108 to allow for the bottom-loading of the first portion 800 of the head 106 (e.g., which is coupled to the shank 104) into the housing 108. For example, the first portion 800 of the head 106 is bottom loaded into the second portion 802 of the head 106, which is pre-assembled within the head cavity 116 of the housing 108. The first portion 800 and the second portion 802 are couplable together via interlocking features (e.g., complementary threading or other interlocking components), such that insertion of the first portion 800 into the second portion 802 that is pre-assembled within the head cavity 116 of the housing 108 and engagement of the interlocking features prevents removal of the head 106 from the distal end of the housing 108.

Proximate to the head cavity 116 (e.g., within the base 110, the housing 108 includes one or more pin apertures 804 that are each operable to receive a pin 806. In some embodiments, the pin 806 includes a first portion 808 and a second portion 810 joined at a shear point 812. When the first portion 808 and the second portion 810 are joined at the shear point 812, the second portion 802 of the head 106 and the housing 108 (and thus the first portion 800 of the head 106 and the shank 104 of the fastener 102, when engaged with the second portion 802 of the head 106) are immobile relative to one another, preventing polyaxial movement of the housing 108 about the head of the fastener 102.

A pre-determined amount of force applied to a tool engagement feature 120 of the housing 108 and/or a tool engagement feature 130 in the head 106 shears (or otherwise breaks) the pin 806 into the first portion 808 and the second portion 810. In some configurations, the shear point 812 is pre-fabricated in the pin 806 prior to insertion of the pin 806 into the pin aperture 804 of the housing 108 and into a pin aperture 814 in the second portion 802 of the head 106 (or when making contact with a surface of the second portion 802 of the head 106, in general). In other configurations, the pre-determined amount of force is sufficient to shear the pin 806 without need for the pre-fabricated shear point 812.

It is noted that the pre-determined amount of force necessary to shear (or otherwise break) the pin 806 may be set as an indication of the correct amount of torque being applied during installation. In one non-limiting example, such as in minimally invasive systems where visual identification is limited, the surgeon may tighten the housing 108 onto the fastener 102 with the shank 104 and the first portion 800 of the head 106 unless the pin 806 breaks and provides the surgeon tactile and/or aural indication that the correct torque has been achieved without need for additional visual identification.

In embodiments, an exterior surface 816 of the piston 124 and/or an interior surface 818 within the housing 108 includes a taper from a proximal end to a distal end along the longitudinal axis through the housing 108. For example, the taper of the surface 816 and/or the surface 818 may be between approximately 0 degrees and approximately 10 degrees. In some instances, the taper of the surface 816 and/or the surface 818 may be approximately 2 degrees. The inclusion of the taper on the surface 816 and/or the surface 818 provides an additional locking to reduce or prevent polyaxial movement of the piston 124 relative to the housing 108.

In embodiments, one or more of the tool engagement features 120 of the housing 108 include a pass-through aperture 820 for a tool pin 822 that interfaces with the piston 124. For example, the piston 124 includes a pin aperture 824 that receives the tool pin 822. For instance, the tool pin 822 may be affixed (e.g., welded, glued with an adhesive, secured via fasteners or interlocking assemblies, or the like) into the pin aperture 824 in the piston 124.

Optionally, the tool pins 822 protrude at least a portion into the tool engagement feature 120 in the one or more arms 112 of the housing 108, such that the tool pins 822 are accessible by a tool. Engaging the tool engagement features 120 with the tool thus will also engage the tool pins 822 and, in doing so, reduce or prevent movement of the housing 108 (and piston 124 within the channel 114 of the housing 108) and restrict polyaxial movement of the housing 108.

It is noted that the pass-through apertures 820 may be increased in dimension (e.g., of greater diameter/length and/or width) than the diameter of the tool pin 822, to allow for translation of the piston 124 between proximal and distal positions within the housing 108. This is beneficial where, in one-non-limiting example, the tool pin 822 (and corresponding apertures 820, 824) are combined with biasing members 200, 500, 600 and the securing of the ring 136 within the head 106. In this regard, it should be understood that the tool pin 822 configuration of the piston 124 may be combined with the biasing members 200, 500, 600 instead of (or in addition to) the shearable pins 806, without departing from the scope of the present disclosure.

It is contemplated, however, that the tool pins 822 may instead be pins at the housing-contact ends of the tool that are configured to engage the tool engagement features 120 on the housing 108, without departing from the scope of the present disclosure. For example, the tool pins 822 on the housing-contact ends of the tool may be insertable into the pin apertures 824 in the piston 124 while other portions of the housing-contact ends of the tool engage the tool engagement feature 120. In this regard, the tool pins 822 being affixed into the piston 124 should be considered optional within a housing assembly of the modular screw 100, for purposes of the present disclosure.

In addition, it is contemplated that a stabilization member (e.g., stabilization member 1010, as illustrated in FIG. 10) positioned within the channel 114 may provide a force to reduce or prevent movement of the housing 108 (and piston 124 within the channel 114 of the housing 108) and restrict polyaxial movement of the housing 108, either in addition to or instead of the tool pins 822. As such, it should be understood that the tool pins 822 (and corresponding apertures 820, 824) may be considered optional in the modular screw 100, for purposes of the present disclosure.

In this regard, the housing 108, the second portion 802 of the head 106 and engaging pins 806, and optionally the piston 124 with the tool pins 822, are fixed together and immovable relative to one another (e.g., holding a pre-determined angle and/or orientation, including as determined and pre-set based on specific implantation parameters for a particular patient) as a pre-assembly 826 (or housing assembly 826). The pre-assembly 826 may be fabricated and provided prior to engagement of the pre-assembly 826 and the first portion 800 of the head 106 of the fastener 102 (e.g., via the second portion 802 of the head 106 of the fastener 102).

It is noted that the pre-assembly 826 may be installed on the shank 104 with the first portion 800 of the head 106 of the fastener 102 after insertion, such that the shank 104 with the first portion 800 of the head 106 of the fastener 102 is first installed in the vertebrae or other holding device and the pre-assembly 826 is then installed. For example, the shank 104 with the first portion 800 of the head 106 of the fastener 102 may be installed using the tool engagement feature 130 in the first portion 800 of the head 106 of the fastener 102, and the pre-assembly 826 may be installed using the tool engagement feature 120 on the housing 108 (including, optionally, to shear the pin 806 for polyaxial movement of the housing 108 relative to the head 106 of the fastener 102 during installation). It is noted that a counter-torque may be applied to the fastener 102 via the tool engagement feature 130 during the installation of the housing 108 and/or optional shearing of the pins 806.

Alternatively, the pre-assembly 826 and the shank 104 with the first portion 800 of the head 106 of the fastener 102 may be assembled as the modular screw 100 prior to insertion of the modular screw 100. Here, the tool engagement feature 130 in the first portion 800 of the head 106 of the fastener 102 or the tool engagement feature 120 on the housing 108 is used to install the modular screw 100, and the tool engagement feature 120 on the housing 108 is optionally used to shear the pin 806 to allow for polyaxial movement of the housing 108 relative to the head 106 of the fastener 102. It is noted that a counter-torque may be applied to the fastener 102 via the tool engagement feature 130 during the installation of the modular screw 100 and/or optional shearing of the pins 806.

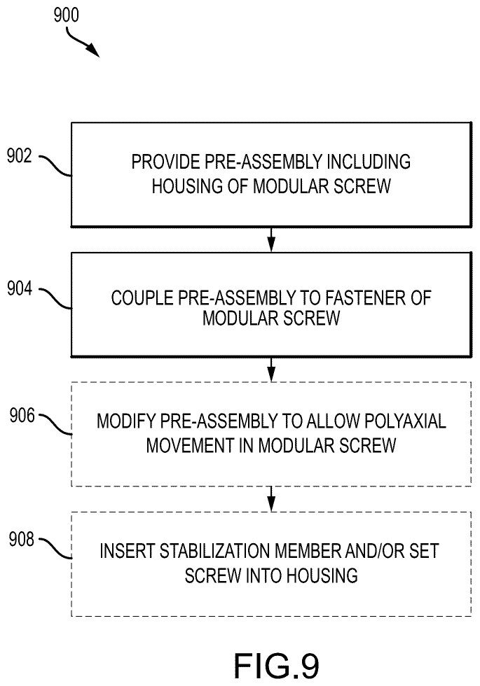

FIG. 9 is a method or process flow diagram 900 illustrating the installation and operation of a vertebral fixation system including the modular screw 100, in accordance with one or more embodiments or the present disclosure. While a general order for the operations of the method or process is shown in FIG. 9, the method or process can include more or fewer operations or can arrange the order of the operations differently (including simultaneously, substantially simultaneously, or sequentially) than those shown in FIG. 9. It is noted that the method or process shall be explained with reference to the components, devices, subassemblies, environments, etc. described in conjunction with FIGS. 8A-8B. For example, it is noted that the embodiments as illustrated in FIGS. 8A-8B should be understood as reading on the embodiments described with respect to FIG. 9, and vice versa, without departing from the scope of the present disclosure.

In embodiments, a pre-assembly of a modular screw is provided 902. The pre-assembly 826 includes the housing 108, one or more pins 806 that engage the housing 108 and the second portion 802 of the head 106 of the fastener 102. Optionally, the pre-assembly 826 additionally includes one or more tool pins 822 that engage the piston 124 within the channel 114 of the housing 108.

In embodiments, the pre-assembly is coupled 904 to a fastener. For example, the shank 104 and the first portion 800 of the head 106 of the fastener 102 may be installed in a vertebrae, and the pre-assembly 126 may then be coupled to the first portion 800 (e.g., via engagement between the first portion 800 on the shank 104 and the second portion 802 within the pre-assembly 826) to form the modular screw 100. By way of another example, the pre-assembly 826 may be coupled to the shank 104 and the first portion 800 of the head 106 of the fastener 102 (e.g., via engagement between the first portion 800 on the shank 104 and the second portion 802 within the pre-assembly 826) to form the modular screw 100, and the modular screw 100 is installed within the vertebrae.

In embodiments, the pre-assembly is modified 906 to allow for polyaxial movement in the modular screw. For example, a pre-determined amount of force applied to the housing 108 may cause the pins 806 to shear or otherwise break, freeing the second portion 802 of the head 106 (and thus the fastener 102) from being interlocked with the housing 108 and allowing for polyaxial movement of the housing 108 about the head 106 of the fastener 102. It is noted that this operation may be optional, however, such as where polyaxial movement of the housing 108 about the head 106 of the fastener 102 is not desirable.

In embodiments, a stabilization member and/or a set screw is inserted 908 into the housing. In embodiments, the stabilization member (e.g., stabilization member 1010, as illustrated in FIG. 10) is inserted into the channel 114, and optionally at least partially contacts the piston 124. In addition (or in the alternative), the set screw (e.g., a set screw 830, as illustrated in FIGS. 8A and 8B) or other interlocking component is inserted into the channel 114, where the set screw or other interlocking component is configured to engage the interlocking mechanism 132 of the housing 108. For example, the engaging the interlocking mechanism 132 may secure the set screw or other interlocking component within the housing 108 to hold the stabilization member within the channel 114. It is noted that the insertion of the stabilization member and/or the set screw is optional, such as where additional modular screws 100 are installed in additional vertebrae prior to insertion of the stabilization member and/or the set screw in the implanted modular screw 100, to allow for the positioning and securing of multiple modular screws 100 with the stabilization member and/or respective set screws together.

In embodiments, one or more of the providing 902, coupling 904, modifying 906, and insertion 908 may be performed for implantation of additional modular screws 100 in additional vertebrae.

Although embodiments of the method or process 900 include individual insertion of components of the modular screw 100 into a patient, it is noted that the vertebral fixation system including the modular screw 100 may be combined using at least operations 902, 904, 906, 908 exterior to and without contact with the human body. For example, the components may be combined during testing and/or experimentation, clinical studies and/or training seminars for how to use the vertebral fixation system including the modular screw 100, or another scenario in which the vertebral fixation system is not yet coupled to a patient but instead where the fastener 102 is inserted into a holding device (e.g., hard surface, ballistic gel, artificial or removed osseous structure, or the like) that mimics the installation surface of a vertebra.

FIG. 10 illustrates a variation of the modular screw 100, in accordance with embodiments of the present disclosure.

In embodiments, the modular screw 100 includes the fastener 102 with the shank 104 and the head 106. The modular screw 100 includes the housing 108, which is able to receive a stabilization member 1010.

The shank 104 and the head 106 are separate components, with the shank 104 including a bore 1000 into which a shaft 1002 of the head 106 may be inserted. For example, the bore 1000 and the shaft 1002 may engage via an interlocking assembly 1004 (e.g., complementary threading, or other interlocking components) with components in the bore 1000 and on the shaft 1002.

To reduce the possibility of impaction of biologic material within the bore 1000 of the shank 104, the shank 104 and the head 106 may have respective contoured surfaces 1006, 1008 that the location of connection between the shank 104 and the head 106. Although not shown, a gasket or other seal may additionally be positioned at the joining of the contoured surfaces 1006, 1008 (or provided as an alternative to the needing of the contoured surfaces).

Although embodiments are directed to the shank 104 having the bore 1000 and the head 106 having the shaft 1002, it is noted that the shank 104 may have a shaft onto which a bore within the head 106 may be coupled, without departing from the scope of the present disclosure. In this regard, the bore 1000 of the shank 104 may generally be called a first section of the fastener 102, and the shaft 1002 of the head 106 may generally be called a second section of the fastener 102.

It is noted that the fastener 102 including the shank 104 with the bore 1000 and the head 106 with the shaft 1002 may be usable any of the embodiments of the modular screw 100 in FIGS. 1A-1D, 2A-2F, 3A-3D, 4A-4E, 5, 6A-6E, and/or 8A-8B without departing from the scope of the present disclosure.

In this regard, advantages of the present disclosure include, but are not limited to, a modular screw including a fastener with a head, and with a shank that is threadable into a vertebra. Advantages of the present disclosure also include, but are not limited to, the head being able to receive a housing after the fastener is inserted into the vertebrae, which couples to the head following the head passing through an aperture in a distal end of the housing (i.e., such that the housing is bottom-loaded with the head of the fastener). After the head is inserted into the distal end of the housing, components within the housing engage with the head and prevent the removal of the head from the distal end of the housing. In this regard, the complexity of the vertebral fixation system is reduced, as the need for a tool that is able to interface with the housing and/or pass through the housing to turn the fastener into the vertebrae is reduced or eliminated. This additionally allows for a smaller tool, and further allows for a reduced size of surgical site and impact on a patient, especially in minimally invasive systems.

Advantages of the present disclosure are further directed to biasing members utilized to actuate components within the housing, such as a piston and a ring. In particular, engagement with a biased piston to cause a transition toward a proximal end of the housing releases a ring that transitions to a position within the housing where the ring can engage with the head of the fastener. Thus, removal of the fastener from the distal end of the housing is prevented by the ring.

Advantages of the present disclosure are further directed to the spring being integrally formed with the piston, and/or are directed to the piston being a single-piece component or a multi-piece component or assembly.

Advantages of the present disclosure are further directed to a pre-assembled housing assembly including the housing, the piston, a second portion of the head of the fastener that is able to receive a first portion of the head of the fastener, and a pin that secures the second portion of the head of the fastener to the housing. The pin prevents polyaxial movement of the housing about the head of the fastener, but can be sheared or otherwise broken with the application of a pre-determined amount of force to allow for polyaxial movement of the housing about the head of the fastener. Advantages of the present disclosure are further directed to reduce or preventing polyaxial movement of the piston relative to the housing, including by contact between interior housing and exterior piston surfaces that may be tapered and/or via contact by an installation tool to tool pins that pass through the housing and are coupled to the piston

While various embodiments of the present disclosure have been described in detail, it is apparent that modifications and alterations of those embodiments will occur to those skilled in the art. However, it is to be understood that such modifications and alterations are within the scope and spirit of the present disclosure, as set forth in the following claims. Further, the disclosure described herein is capable of other embodiments and of being practiced or of being carried out in various ways. It is to be understood that the phraseology and terminology used herein is for the purpose of description and should not be regarded as limiting.

What is claimed is:

1. A modular screw for a vertebral fixation system, comprising:
a housing assembly, comprising:
a housing including a base and at least one arm, wherein the base and the at least one arm define a head cavity, a piston cavity, and a channel able to receive a stabilization member;
a piston installed within the piston cavity and at least partially extending into the head cavity;
a ring installed within the head cavity; and
one or more biasing members that bias the piston toward the ring and into the head cavity; and
a fastener comprising a shank and a head, wherein the head is insertable into the head cavity via a bottom-loading of the head into the head cavity through a head aperture at a distal end of the housing.

2. The modular screw of claim 1, wherein the one or more biasing members causes the piston to be biased toward the distal end of the housing absent an application of force to the piston by the head of the fastener,
wherein the ring is held in a first position by the piston prior to an application of force by the head on the piston,
wherein the ring is released by the piston following an application of force by the head on the piston and engages the head, and wherein the ring engaging the head prevents removal of the head from the distal end of the housing.

3. The modular screw of claim 1, wherein the one or more biasing members comprises a pin extending into the housing and a spring that engages the piston.

4. The modular screw of claim 1, wherein the one or more biasing members comprises a spring inserted within the housing and engages the piston, wherein the spring is inserted at an oblique angle relative to a longitudinal axis through the housing.

5. The modular screw of claim 1, wherein the piston comprises:
a first portion to engage with the one or more biasing members; and
a second portion to engage with at least one of the ring and the head.

6. The modular screw of claim 1, wherein the ring is a c-ring.

7. The modular screw of claim 1, wherein the modular screw is configured for polyaxial movement of the housing relative to the head of the fastener.

8. The modular screw of claim 1, wherein the modular screw is configured for unidirectional movement of the housing relative to the head of the fastener.

9. The modular screw of claim 8, wherein the ring includes one or more trunnions when the modular screw is configured for unidirectional movement.

10. The modular screw of claim 1, further comprising:
a locking assembly, comprising:
one or more members within respective member channels in the housing; and
one or more pins that correspond to a particular member of the one or more members,
wherein a force applied to a particular member of the one or more members is transferred through the corresponding pin of the one or more pins to the piston, and
wherein the force being applied to the piston prevents rotation in one or more directions of the head of the fastener relative to the housing.

11. The modular screw of claim 1, being usable as a component of the vertebral fixation system with at least one of a set screw and a stabilization member that are each insertable into the housing.

12. A modular screw for a vertebral fixation system, comprising:
a fastener comprising a shank and a head, the head including a first portion; and
a housing assembly, comprising:
a housing including a base and at least one arm, wherein the base and the at least one arm define a head cavity, a piston cavity, and a channel able to receive a stabilization member;
a piston installed within the piston cavity and at least partially extending into the head cavity;
a second portion of the head of the fastener within the head cavity; and
a pin able to secure the second portion of the head of the fastener at a particular orientation within the head cavity relative to the housing,
wherein the first portion of the head is insertable into the second portion of the head within the housing assembly via a bottom-loading of the first portion of the head into the head cavity through a head aperture at a distal end of the housing,
wherein polyaxial movement of the housing about the head of the fastener is reduced by the pin when the pin engages the housing and the second portion of the head of the fastener, and
wherein the pin is able to be sheared at a shear point with a pre-determined amount of force to allow for polyaxial movement of the housing about the head of the fastener.

13. The modular screw of claim 12, wherein the base and the at least one arm of the housing further define a piston cavity, wherein the housing assembly further comprises a tool pin in engagement with a pin aperture in the piston, and wherein the tool pin is located within a tool engagement feature in the at least one arm of the housing.

14. The modular screw of claim 13, wherein the tool pin is affixed to the pin aperture of the piston.

15. The modular screw of claim 13, wherein at least one of an interior surface of the housing and an exterior surface of the piston is tapered to reduce polyaxial movement of the piston relative to the housing.

16. A method, comprising:
providing a housing assembly and a fastener of a modular screw, the housing assembly comprising a piston within a housing including a base and at least one arm that define a head cavity, a piston cavity, and a channel able to receive a stabilization member, the fastener comprising a shank and a head; and
inserting at least a portion of the head of the fastener into the head cavity of the housing via bottom-loading into a head aperture at a distal end of the housing,
wherein engagement of interlocking features within the head cavity of the housing post-insertion of the at least a portion of the head of the fastener into the head cavity of the housing prevents removal of the head from the distal end of the housing.

17. The method of claim 16, further comprising:
actuating the piston with the head toward a proximal end of the housing relative to a position pre-set by one or more biasing members that engage with and bias the piston toward the distal end of the housing, wherein the actuating of the piston with the head releases a ring installed within the head cavity and into the head cavity, and wherein the released ring engages with the head to prevent removal of the head from the distal end of the housing.

18. The method of claim 16, further comprising:
providing a pre-assembly of the modular screw, the pre-assembly including the housing, the piston, a second portion of the fastener that is able to receive and engage with a first portion of the fastener, and a pin able to secure the second portion of the head of the fastener at a particular orientation within the head cavity relative to the housing; and
coupling the first portion of the head of the fastener into the second portion of the head of the fastener within the head cavity of the housing via threading that prevents removal of the head from the distal end of the housing.

19. The method of claim 18, further comprising:
applying a pre-determined amount of force to the modular screw to shear the pin at a shear point and allow for polyaxial movement of the housing about the head of the fastener.

20. The method of claim 18, further comprising:
engaging a tool pin coupled to the piston and accessible via a tool engagement feature in the at least one arm of the housing, wherein engagement of the tool pin prevents polyaxial movement of the piston relative to the housing.
